(19) United States
(12) Patent Application Publication
(10) Pub. No.: US 2026/0102051 A1
(43) Pub. Date: Apr. 16, 2026

(54) ENDOSCOPE CONTROL DEVICE, ENDOSCOPE SYSTEM, AND ENDOSCOPE CONTROL METHOD

(71) Applicant: OLYMPUS MEDICAL SYSTEMS CORP., Tokyo (JP)

(72) Inventors: Jun HANE, Tokyo (JP); Hiromasa FUJITA, Tokyo (JP); Kensuke MIYAKE, Tokyo (JP)

(73) Assignee: OLYMPUS MEDICAL SYSTEMS CORP., Tokyo (JP)

(21) Appl. No.: 19/421,967

(22) Filed: Dec. 16, 2025

Related U.S. Application Data
(63) Continuation of application No. PCT/JP2023/022765, filed on Jun. 20, 2023.

(51) Int. Cl. A61B 1/00; G06T 7/00

(52) U.S. Cl. CPC A61B 1/00009; A61B 1/00045; A61B 1/00124; G06T 7/0016; G06T 2207/10068; G06T 2207/30096

(57) ABSTRACT

An endoscope control device includes a connection interface, and one or more processors including hardware that are configured to acquire, via the connection interface, endoscope information and image information of an observation target region at a plurality of timings at which an endoscope is positioned in the observation target region, and perform lesion identification determinations each determining whether lesions observed at the plurality of timings are identical to each other, thereby generating comparison information including one or more results derived from the lesion identification determinations. The lesion identification determinations include a first lesion identification determination based on the endoscope information, a second lesion identification determination based on the image information, and a third lesion identification determination based on results of the first lesion identification determination and the second lesion identification determination.

### CROSS REFERENCE TO RELATED APPLICATION

This application is a continuation of International Application No. PCT/JP2023/022765, filed on Jun. 20, 2023, the entire contents of which are incorporated herein by reference.

### FIELD

The present disclosure relates to an endoscope control device that is used at the time of a colonoscopy, for example, an endoscope system, and an endoscope control method.

### BACKGROUND

Conventionally, endoscopes have been widely used, for example, in the medical field, the industrial field, and the like. Medical endoscopes that are used in the medical field have a function of inserting an insertion portion provided with an image pickup unit into a body of a subject such as a living body, and acquiring observation target images or the like of a lesion part, etc., inside an organ, or the like. The images thus acquired are used for an image diagnosis or the like for performing an observation or an examination of the lesion part, and the like. Conventional medical endoscopes include, for example, colonoscopes designed for colonoscopy and the like, which primarily observe, examine, and treat an inside of a lumen of the large intestine, and such devices are put into practical use and widely available.

In general, in the colonoscopy, an insertion portion of an endoscope is inserted through the anus, the rectum, and the colon to the vicinity of the intestinal cecum, and endoscopic images acquired while removing the insertion portion are used to screen a lesion part and the like. If the lesion part and the like are confirmed, a detailed examination of the lesion part and the like are performed using the endoscopic images.

Here, for example, various information (in particular, position information, state information, and the like) related to the confirmed lesion part and the like is recorded. In addition, depending on a state of the confirmed lesion part and the like, various treatments and the like may be performed on the lesion part and the like. For example, a treatment such as taking a biological tissue from the vicinity of the confirmed lesion part and the like is performed for a pathological diagnosis or the like.

In the conventional colonoscopy, various proposals have been made for means of recording various information regarding a lesion part and the like, in Japanese U.S. Pat. No. 6,749,473, for example.

The endoscope system disclosed in the above-mentioned Japanese U.S. Pat. No. 6,749,473 and the like detects a region (observation target region) where a lesion part and the like exist in a lumen using acquired endoscopic images, and acquires position information of the target region. Simultaneously, the endoscope system acquires shape information of an insertion portion of an endoscope which is inserted into the lumen, and acquires an endoscope insertion shape image using the shape information of the insertion portion. Then, a marking image is generated on the endoscope insertion shape image by marking a position corresponding to the position information of the target region, and the marking image is displayed on a display device.

Various information regarding the lesion part and the like recorded at this time, the insertion portion shape information, the marking image, and the like are used as information for identifying the lesion part and the like, during a re-examination to be performed at a later date, during a treatment to be performed at a later date, or the like.

Here, the re-examination includes, for example, when no treatment or the like was performed on the lesion part and the like that were confirmed in this examination, a subsequent examination for a follow-up observation, or the like, of observing changes in conditions of the confirmed lesion part and the like. In addition, for example, when a treatment or the like was performed on the lesion part and the like that were confirmed in this examination, a subsequent examination and the like, for the follow-up observation of observing changes in conditions of traces and the like after the treatment are included.

### SUMMARY

According to aspects of the present disclosure, an endoscope control device is provided, which includes a connection interface and one or more processors including hardware. At least one of the one or more processors is configured to acquire, via the connection interface, endoscope information and image information of an observation target region at a plurality of timings at which an endoscope is positioned in the observation target region. Further, at least one of the one or more processors is configured to perform lesion identification determinations each determining whether lesions observed at the plurality of timings are identical to each other, thereby generating comparison information including one or more results derived from the lesion identification determinations. The lesion identification determinations include a first lesion identification determination based on the endoscope information. The lesion identification determinations further include a second lesion identification determination based on the image information. The lesion identification determinations further include a third lesion identification determination based on results of the first lesion identification determination and the second lesion identification determination.

According to aspects of the present disclosure, further provided is an endoscope system that includes an endoscope and an endoscope control device. The endoscope control device includes a connection interface and one or more processors including hardware. At least one of the one or more processors is configured to acquire, via the connection interface, endoscope information and image information of an observation target region at a plurality of timings at which an endoscope is positioned in the observation target region. Further, at least one of the one or more processors is configured to perform lesion identification determinations each determining whether lesions observed at the plurality of timings are identical to each other, thereby generating comparison information including one or more results derived from the lesion identification determinations. The lesion identification determinations include a first lesion identification determination based on the endoscope information. The lesion identification determinations further include a second lesion identification determination based on the image information. The lesion identification determinations further include a third lesion identification determination based on results of the first lesion identification determination and the second lesion identification determination.

According to aspects of the present disclosure, further provided is an endoscope control method implementable on one or more processors of an endoscope control device. The endoscope control method includes acquiring endoscope information and image information of an observation target region at a plurality of timings at which an endoscope is positioned in the observation target region. The endoscope control method further includes performing lesion identification determinations each determining whether lesions observed at the plurality of timings are identical to each other, thereby generating comparison information including one or more results derived from the lesion identification determinations. The lesion identification determinations include a first lesion identification determination based on the endoscope information. The lesion identification determinations further include a second lesion identification determination based on the image information. The lesion identification determinations further include a third lesion identification determination based on results of the first lesion identification determination and the second lesion identification determination.

### BRIEF DESCRIPTION OF THE DRAWINGS

FIG. 1 is a schematic configuration diagram showing the entirety of an endoscope system including an endoscope control device of an embodiment of the present disclosure;

FIG. 2 is a block configuration diagram schematically showing the entirety of the endoscope system including the endoscope control device of the embodiment of the present disclosure and schematically showing an internal configuration of the endoscope control device;

FIG. 3 is a conceptual diagram showing a configuration of a part of an endoscope device (endoscope insertion portion) included in the endoscope system of FIG. 1;

FIG. 4 shows a first display example of a qualitative display form of lesion position related information generated by the endoscope control device of the embodiment of the present disclosure;

FIG. 5 shows a second display example of the qualitative display form of the lesion position related information;

FIG. 6 shows a third display example of the qualitative display form of the lesion position related information;

FIG. 7 shows a fourth display example of the qualitative display form of the lesion position related information;

FIG. 8 shows one example of a quantitative display form of the lesion position related information generated by the endoscope control device of the embodiment of the present disclosure;

FIG. 9 shows a first display example of lesion position comparison information generated by the endoscope control device of the embodiment of the present disclosure;

FIG. 10 shows a second display example of the lesion position comparison information;

FIG. 11 shows a third display example of the lesion position comparison information;

FIG. 12 shows a fourth display example of the lesion position comparison information;

FIG. 13 shows a fifth display example of the lesion position comparison information;

FIG. 14 shows a sixth display example of the lesion position comparison information;

FIG. 15 shows an example of a qualitative display of two pieces of lesion position related information for generating a seventh display example of the lesion position comparison information;

FIG. 16 shows the seventh display example of the lesion position comparison information;

FIG. 17 is a conceptual diagram showing an approach for lesion identification determination based on images;

FIG. 18 shows an eighth display example of the lesion position comparison information;

FIG. 19 shows a ninth display example of the lesion position comparison information;

FIG. 20 shows a tenth display example of the lesion position comparison information;

FIG. 21 shows an eleventh display example of the lesion position comparison information;

FIG. 22 is a block configuration diagram showing a schematic configuration of a first modification example of the endoscope system including the endoscope control device of the embodiment of the present disclosure;

FIG. 23 is a block configuration diagram showing the schematic configuration of the first modification example of the endoscope system;

FIG. 24 is a flowchart showing operation when acquiring examination information, insertion portion shape and arrangement information, or the like among operations of the endoscope system of the embodiment of the present disclosure;

FIG. 25 is a flowchart showing operation when generating comparison information in real-time processing among the operations of the endoscope system of the embodiment of the present disclosure; and

FIG. 26 is a flowchart showing operation when generating the comparison information in post-processing among the operations of the endoscope system of the embodiment of the present disclosure.

### DESCRIPTION OF EMBODIMENTS

Hereinafter, the present disclosure will be described by illustrated embodiments. The respective drawings used in the following description are schematically shown, and in order to show each of the constituent elements in a size recognizable in the drawings, there is a case where the dimensional relationship, scales, etc., of the respective members are made different for each of the constituent elements. Therefore, the present disclosure is not limited only to the illustrated forms with regard to the number, the shapes, the ratio of the sizes, the relative positional relationship, etc., of the respective constituent elements illustrated in each of the drawings.

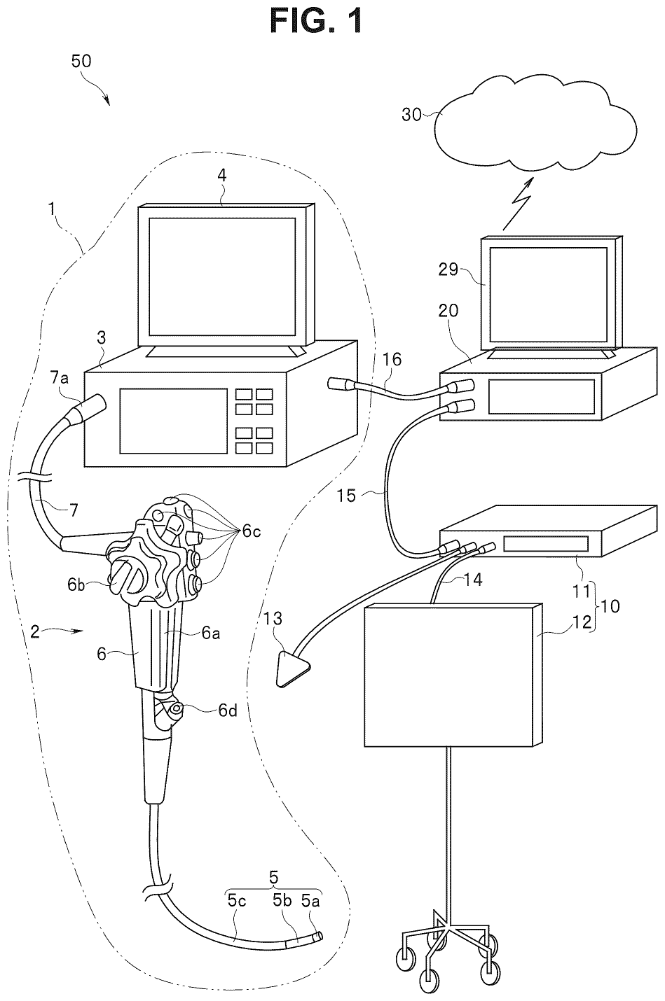

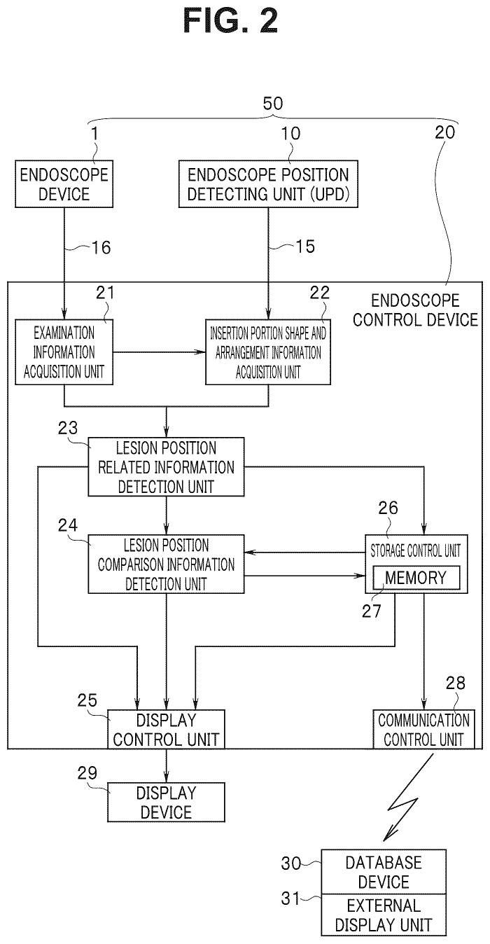

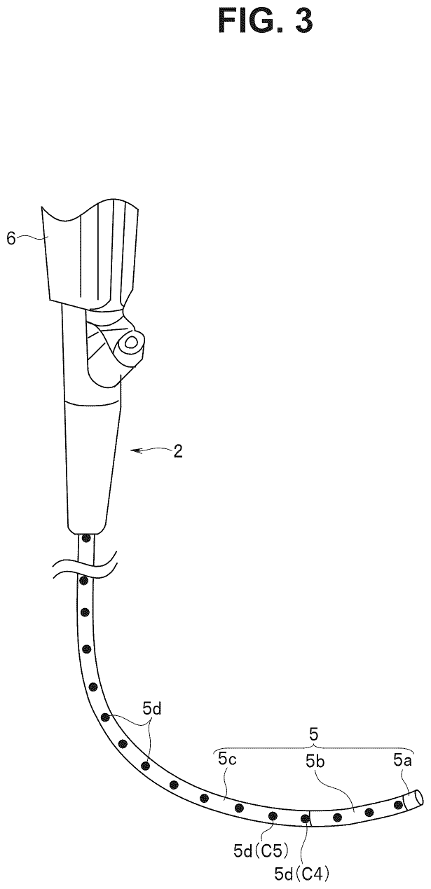

A schematic configuration of the entirety of an endoscope system including an endoscope control device of an embodiment of the present disclosure will now be described with reference to FIG. 1 to FIG. 3. FIG. 1 is a schematic configuration diagram showing the entirety of the endoscope system including the endoscope control device of the embodiment of the present disclosure. FIG. 2 is a block configuration diagram schematically showing the entirety of the endoscope system of FIG. 1 and schematically showing an internal configuration of the endoscope control device. FIG. 3 is a conceptual diagram showing a configuration of a part of the endoscope device (endoscope insertion portion) included in the endoscope system of FIG. 1 and FIG. 2.

As shown in FIG. 1 and FIG. 2, an endoscope system 50 including an endoscope control device 20 of the embodiment of the present disclosure includes an endoscope device 1, an endoscope position detecting unit (hereinafter, abbreviated as UPD) 10, the endoscope control device 20, a display device 29, a database device 30, and the like.

Among these, basic configurations of the endoscope device 1, the UPD 10, the display device 29, and the database device 30 are respectively the same as those of conventionally known devices of the same type. Therefore, the configurations of these devices are described briefly, and detailed descriptions are omitted.

The endoscope device 1 includes an endoscope 2, a video processor 3, an endoscope monitor device 4, and the like, as shown in FIG. 1. The endoscope device 1 illustrated in the present embodiment is assumed to be a colonoscope device for performing a colonoscopy to observe an inside of the large intestine of a subject (patient).

The endoscope 2 includes an insertion portion 5, an operation portion 6, a universal cord 7, and the like.

The insertion portion 5 is a constituent member to be inserted into a body of the subject such as a living body. The insertion portion 5 is formed by continuously connecting a distal-end constituting portion 5a, a bending portion 5b, and a flexible tube portion 5c in this order from a distal end side. The insertion portion 5 is formed in a substantially elongated tubular shape as a whole. The operation portion 6 is connected to a proximal end side of the insertion portion 5.

In addition, although details are omitted, the endoscope 2 is formed so that an endoscope treatment instrument (not shown) can be inserted therethrough. Specifically, a treatment instrument insertion channel, which is a conduit for inserting the treatment instrument (not shown), is provided so as to insert through the insertion portion 5 between the distal end and the proximal end thereof (not shown).

Furthermore, various constituent members (not shown) such as an image pickup unit and an illumination unit are provided inside the distal-end constituting portion 5a. Here, the image pickup unit is an electronic device unit including a photoelectronic conversion element, an optical lens and the like for acquiring image information (still images and moving images) of an observation target object (such as a lumen inner wall of an organ such as large intestine, for example) inside the subject. The illumination unit is a constituent unit including an optical element, or the like, which emits a light flux guided from a light source device (not shown), described later, from a distal end surface of the distal-end constituting portion 5a toward the front to illuminate an observation target region including the lesion part and the like in the subject.

The operation portion 6 is connected to the proximal end of the insertion portion 5. The operation portion 6 includes an operation portion main body 6a, a bending operation knob 6b, a plurality of operation members 6c, a treatment instrument insertion port 6d, and the like.

The operation portion main body 6a is in a substantially box-shape as a whole, and forms a grasping portion to be grasped by a user, such as an operator, of the endoscope 2. The insertion portion 5 is extended from the operation portion main body 6a, as described above.

The bending operation knob 6b and the plurality of operation members 6c are operation members for performing various operations of the endoscope 2. In particular, the bending operation knob 6b is used when operating a bending state or a bending direction of the bending portion 5b, or fixing/releasing the bending state of the bending portion 5b. The bending operation knob 6b and the plurality of operation members 6c are respectively provided at predetermined positions on an outer surface of the operation portion main body 6a.

The treatment instrument insertion port 6d is provided at a predetermined position near a distal end of the operation portion main body 6a. The treatment instrument insertion port 6d is a proximal end side opening of the treatment instrument insertion channel (not shown) of the insertion portion 5. The treatment instrument insertion channel is connected to a distal end opening (not shown) of the distal-end constituting portion 5a on a distal end side thereof. With this configuration, a distal end of the treatment instrument (not shown) inserted from the treatment instrument insertion port 6d can protrude outward from the distal end opening of the distal-end constituting portion 5a.

Note that the endoscope 2 of the endoscope device 1 included in the endoscope system 50 of the present embodiment includes a configuration for corresponding to the UPD 10. Specifically, for example, the endoscope 2 includes a plurality of magnetic coils 5d inside the insertion portion 5 (not shown in FIG. 1, see FIG. 3).

Specifically, as shown in FIG. 3, the plurality of magnetic coils 5d are arranged along an insertion axis direction (longitudinal direction) of the insertion portion 5 at a predetermined distance (an interval of approximately 10 cm (centimeters), for example). Each of the magnetic coils 5d has a configuration that generates a magnetic field when an electric current is supplied.

Note that the reference sign C4 in FIG. 3 indicates a magnetic coil corresponding to a most distal end position of the flexible tube portion 5c (near the boundary with the bending portion 5b). In addition, the reference sign C5 in FIG. 3 indicates a magnetic coil C5 that is located at one position closer to the proximal end than the magnetic coil C4.

Here, the UPD 10 detects respective positions of the plurality of magnetic coils 5d disposed in the insertion portion 5. Then, the UPD 10 acquires information on a three-dimensional shape of the insertion portion 5 based on the detected position information of the plurality of magnetic coils 5d. In this case, the plurality of magnetic coils 5d function as a position sensor for detecting the three-dimensional shape of the insertion portion 5.

Returning to FIG. 1, the universal cord 7 is a tubular member extending from a side of the operation portion main body 6a of the operation portion 6. A scope connector 7a is provided at a distal end of the universal cord 7. The scope connector 7a is connected to a front surface panel of the video processor 3. As described later, various signal transmission cables and optical fiber cables are inserted through the universal cord 7.

The video processor 3 is a control device and a signal processing device including a control circuit, a signal processing circuit, and the like, for controlling the entire endoscope device 1. The control circuit in the video processor 3 receives an operation instruction signal of the operation member 6c of the operation portion 6 of the endoscope 2, and outputs various control signals for driving and controlling the image pickup unit, the light source device, the illumination unit, and the like, for example. In addition, the signal processing circuit receives an image pickup signal from the image pickup unit (not shown) provided inside the distal-end constituting portion 5a of the insertion portion 5 of the endoscope 2, for example, and performs predetermined image signal processing, and the like.

Therefore, the video processor 3 and the image pickup unit are electrically connected to each other via a signal transmission cable (not shown). The signal transmission cable is disposed so as to be inserted from the scope connector 7a to the image pickup unit of the distal-end constituting portion 5a through the universal cord 7, the operation portion 6, and the insertion portion 5. With this configuration, the control signal outputted from the video processor 3, the image pickup signal outputted from the image pickup unit and the like are transmitted between the image pickup unit and the video processor 3 through the signal transmission cable. Note that as one form of the signal transmission cable, for example, a composite cable in which a plurality of cables are bundled and covered with an outer shield, an outer tube, or the like is applied.

In addition, the light source device (not shown) is provided inside the video processor 3. The light source device is a device for supplying illumination light to the illumination unit provided inside the distal-end constituting portion 5a of the insertion portion 5 of the endoscope 2. The illumination light emitted from the light source device is transmitted to the illumination unit of the distal-end constituting portion 5a through the optical fiber cable (not shown) and the like disposed so as to be inserted, from the scope connector 7a, through the universal cord 7, the operation portion 6, and the insertion portion 5. Then the illumination light is transmitted through an illumination lens included in the illumination unit of the distal-end constituting portion 5a, and irradiated toward the observation target region in front of the distal-end constituting portion 5a.

Note that the illumination unit is not limited to the above-described configuration example (the form in which the illumination light from the light source device is transmitted to the distal-end constituting portion 5a through the optical fiber cable, or the like) as one example. For example, a light emitting diode (LED) or the like, as an illumination light source may be provided in the distal-end constituting portion 5a, and a control circuit of the video processor 3 may perform light emission control of the illumination light source (LED).

The video processor 3 is then electrically connected to the endoscope control device 20 of the present embodiment through a connection cable 16. With this configuration, the video processor 3 outputs endoscopy information (sometimes simply referred to as endoscope information) including various information regarding an endoscopic image and a lesion part and the like, to the endoscope control device 20 through the connection cable 16.

The endoscope monitor device 4 is a display device for receiving image signals and the like outputted from the video processor 3, and displaying the endoscopic image and various information and the like in predetermined forms.

Therefore, the endoscope monitor device 4 and the video processor 3 are electrically connected to each other using a video cable (not shown). Note that as a form of the endoscope monitor device 4, for example, a display device configured using a general liquid crystal panel is applied.

The UPD 10 is a device for observing a three-dimensional shape of the insertion portion 5 of the endoscope 2 inserted into the body (specifically, in the lumen such as the large intestine and the like, for example) of the subject. The UPD 10 includes a main body control processor 11, a receiving antenna 12, a reference plate 13, and the like.

The receiving antenna 12 is an antenna device for detecting a magnetic field generated from the plurality of magnetic coils 5d (see FIG. 3) provided inside the insertion portion 5 of the endoscope 2, and outputting a predetermined detection signal to the main body control processor 11. Therefore, the receiving antenna 12 is electrically connected to the main body control processor 11 through a connection cable 14.

The main body control processor 11 is a device including a signal processing circuit and the like for receiving a magnetic field detection signal of the plurality of magnetic coils 5d outputted from the receiving antenna 12, and performing predetermined signal processing. The main body control processor 11 is electrically connected to the endoscope control device 20 through a connection cable 15.

For example, the main body control processor 11 applies a magnetic field strength of each of the plurality of magnetic coils 5d received by the receiving antenna 12, to a predetermined position detection algorithm to estimate a three-dimensional position of each of the plurality of magnetic coils 5d. In addition, the main body control processor 11 performs a curve interpolation, for example, based on the estimated three-dimensional position information of the plurality of magnetic coils 5d, to generate three-dimensional shape information and the like of the insertion portion 5 of the endoscope 2.

The reference plate 13 is a device including a posture sensor or the like for detecting a posture of the subject. Here, as the posture sensor, a three-axis acceleration sensor, a gyro sensor, or the like is applied, for example.

The reference plate 13 is attached to, for example, the abdomen or the like of the subject when the endoscopy is performed. In addition, the reference plate 13 is connected to the main body control processor 11 through a cable. With this configuration, the reference plate 13 acquires a detection signal related to the posture of the subject in the examination. The detection signal acquired by the reference plate 13 is outputted to the main body control processor 11, and the main body control processor 11 performs predetermined signal processing on the detection signal to generate three-dimensional posture information and the like related to the posture of the subject.

Note that as the posture sensor applied to the reference plate 13, a magnetic coil substantially the same as the plurality of magnetic coils 5d applied to the insertion portion 5 of the endoscope 2 can be applied. In such a configuration, the receiving antenna 12 detects a magnetic field generated from the plurality of magnetic coils disposed in the reference plate 13, and outputs the magnetic field as a magnetic field detection signal to the main body control processor 11. The main body control processor 11 receives the magnetic field detection signal, and applies the magnetic field strength of each of the plurality of magnetic coils of the reference plate 13 to a predetermined posture detection algorithm, to generate the three-dimensional posture information and the like related to the posture of the subject.

As described above, the main body control processor 11 is connected to the receiving antenna 12 and the reference plate 13. Then, the main body control processor 11 generates the three-dimensional shape information and the like of the insertion portion 5 based on the three-dimensional position information of the plurality of magnetic coils 5d inputted from the receiving antenna 12. In addition, the main body control processor 11 generates the three-dimensional posture information and the like related to the posture of the subject based on the detection signal related to the posture of the subject inputted from the reference plate 13.

Then, the main body control processor 11 performs signal processing for changing the generated three-dimensional shape information and the like according to a change in the three-dimensional posture information and the like. Specifically, the main body control processor 11 performs processing for changing a three-dimensional endoscope shape in a direction that cancels out the change in the three-dimensional posture information, for example.

This makes it possible to acquire endoscope shape information that can always visualize the endoscope shape from a predetermined viewpoint (a viewpoint and the like when facing the abdomen of the subject, for example), even if the posture of the subject is changed during the endoscopy.

Furthermore, the UPD 10 acquires an insertion length indicating a length dimension of an insertion part when the insertion portion 5 of the endoscope 2 is inserted into the large intestine, and an insertion time indicating an elapsed time after the endoscope 2 is inserted into the large intestine.

The UPD 10 measures the insertion length (from the anus) using, as a reference point, a position of the distal-end constituting portion 5a of the insertion portion 5 at an examination start timing (an insertion timing of the insertion portion 5 of the endoscope 2 into the anus, for example) using the endoscope device 1 by the user of the endoscope (hereinafter, referred to as an operator or the like), for example. At the same time, the elapsed time using the examination start timing as a reference point is measured. A measurement time obtained in this way is the insertion time.

Note that by determining whether the endoscopic image shows inside or outside of the body, the position of the distal end of the insertion portion of the three-dimensional shape information generated by the UPD 10 at the timing of a change from outside the body to inside the body may be estimated as the position of the anus. In addition, at the timing of insertion into the anus, the position of the anus may be estimated by the operator pressing a button of the operation member 6c, or the operator stepping on a foot switch. The estimated position of the anus may then be used as the reference point of the insertion length, and as a measurement start reference point of the insertion time.

In addition, in order to measure the insertion length with high accuracy, it is conceivable that a sensor such as an encoder is installed near the anus of the subject, for example. The UPD 10 detects the position of the anus based on an output signal from the encoder, and detects the insertion length using the position of the anus as the reference point.

Then, the UPD 10 outputs the three-dimensional shape information and the like after correcting the posture based on the three-dimensional posture information and the like, and information to which the information of the insertion length, the insertion time, and the like are added, to the endoscope control device 20 through the connection cable 15.

Note that the UPD 10 detects the endoscope insertion shape using the plurality of magnetic coils incorporated into the insertion portion 5, and the like, but other configurations or different methods may also be used.

The endoscope control device 20 is a control device including an information processing circuit for acquiring various information inputted from the outside, and performing predetermined information processing, a control processing circuit for performing various control such as storage, display, communication, and the like of various information generated by the information processing circuit, and the like.

The endoscope control device 20 includes, as shown in FIG. 2, an examination information acquisition unit 21, an insertion portion shape and arrangement information acquisition unit 22, a lesion position related information detection unit 23, a lesion position comparison information detection unit 24, a display control unit 25, a storage control unit 26, a memory 27, a communication control unit 28, and the like.

The examination information acquisition unit 21 is a configuration circuit or a configuration part into which the endoscopy information and the like acquired mainly by the endoscope device 1 are inputted. The endoscopy information and the like inputted into the examination information acquisition unit 21 is information acquired by the endoscope device 1 during performing the endoscopy. Specifically, the endoscopy information and the like include, in addition to endoscopic image information, various information obtained by processing the endoscopic image, endoscopic image acquisition timing information and the like, for example, operation information of the endoscope, and audio information or video information including an audio of the operator during the examination. The endoscopy information and the like acquired by the examination information acquisition unit 21 are outputted to the lesion position related information detection unit 23.

Note that the above endoscopic image acquisition timing information is information regarding a timing when the endoscopic image is acquired. Specifically, an endoscopic image acquisition timing information is, for example, time information at an image acquisition time point, an elapsed time from an examination start time point to the image acquisition time point, or the like. The endoscopic image acquisition timing information is further outputted from the examination information acquisition unit 21 to the insertion portion shape and arrangement information acquisition unit 22.

The insertion portion shape and arrangement information acquisition unit 22 is a configuration circuit or a configuration part into which various insertion portion shape and arrangement information and the like acquired mainly by the UPD 10 are inputted. The insertion portion shape and arrangement information and the like inputted into the insertion portion shape and arrangement information acquisition unit 22 are the three-dimensional shape information of the insertion portion 5 inserted into the body of the subject during performing the endoscopy, space arrangement information of the insertion portion in the body, and the like.

The insertion portion shape and arrangement information and the like include, for example, insertion length information of the insertion portion 5 at a predetermined timing, three-dimensional position information (xyz coordinates) and orientation information (vector information) of the distal-end constituting portion 5a at a predetermined timing, and the like.

Here, the predetermined timing includes, for example, a timing at which the distal-end constituting portion 5a starts to be inserted into the anus (examination start timing), a timing at which the distal-end constituting portion 5a reaches the vicinity of the intestinal cecum (intestinal cecum reaching timing), an endoscopic image acquisition timing, and the like.

Note that, as described above, the endoscopic image acquisition timing information is information inputted from the examination information acquisition unit 21 to the insertion portion shape and arrangement information acquisition unit 22. The insertion portion shape and arrangement information acquisition unit 22 performs processing for associating the endoscopic image acquisition timing information with the insertion portion shape and arrangement information and the like. With this, the insertion portion shape and arrangement information acquisition unit 22 can generate the insertion portion shape and arrangement information and the like for each endoscopic image acquisition timing.

The insertion portion shape and arrangement information and the like acquired from the insertion portion shape and arrangement information acquisition unit 22 in this way are outputted to the lesion position related information detection unit 23.

The lesion position related information detection unit 23 is a configuration circuit or a configuration part for performing predetermined information processing based on the endoscopy information and the like inputted from the examination information acquisition unit 21, and the insertion portion shape and arrangement information and the like inputted from the insertion portion shape and arrangement information acquisition unit 22, and detecting and generating information regarding the position of each of the plurality of lesion parts and the like (hereinafter, referred to as lesion position related information). The lesion position related information detected and generated by the lesion position related information detection unit 23 is outputted to the lesion position comparison information detection unit 24, the display control unit 25, and the storage control unit 26, as appropriate and necessary.

The lesion position related information is information indicating a correspondence between the positions of the plurality of lesion parts in the body and the insertion shape and the arrangement of the insertion portion 5. The lesion position related information is, for example, displayed using the display device 29 and the like, in a predetermined form such as a qualitative (graphical display, etc.) form or a quantitative (numerical display) form, or in a combination of the both forms, for example.

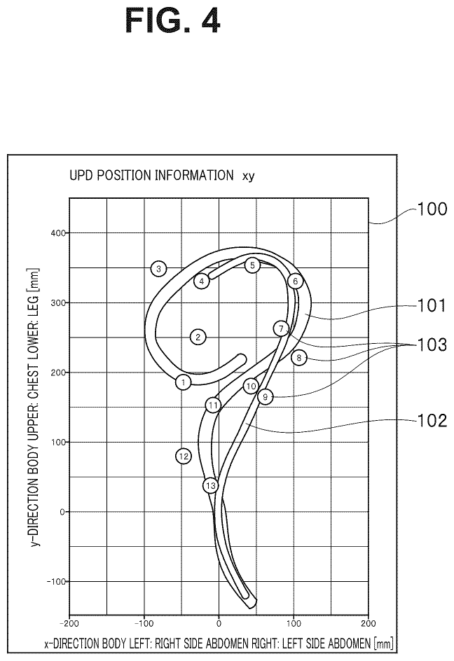

Here, FIG. 4 to FIG. 7 show first to fourth display examples of qualitative display forms of the lesion position related information. As the qualitative display form of the lesion position related information, a form can be considered, for example, in which a graphic display showing the shape and the arrangement of the insertion portion 5 in the subject and a point display (indicated by circles in the figure) indicating positions of the observation target regions (hereinafter abbreviated as target positions) including the plurality of lesion part and the like are combined and superimposed on a graph 100 in a predetermined form.

First, the following are display examples of a graphic showing the shape and the arrangement of the insertion portion 5. For example, the reference sign 101 in FIG. 4 to FIG. 7 is an example of a graphic showing the shape and the arrangement of the insertion portion 5 at a time point (timing) when the distal-end constituting portion 5a reaches the vicinity of the intestinal cecum (hereinafter, referred to as a first insertion shape). There is one graphic data of the first insertion shape for each endoscopy.

Generally, when the distal-end constituting portion 5a reaches the intestinal cecum, a patient as the subject is often in a supine posture with the abdomen facing the ceiling side, and the shape of the insertion portion 5 in the supine posture is shown in FIG. 4 to FIG. 7. In FIG. 4 to FIG. 7, the left and right direction of the subject is taken as an x-direction, the head and foot direction is taken as a y-direction, and the anus position is taken as the origin (0, 0).

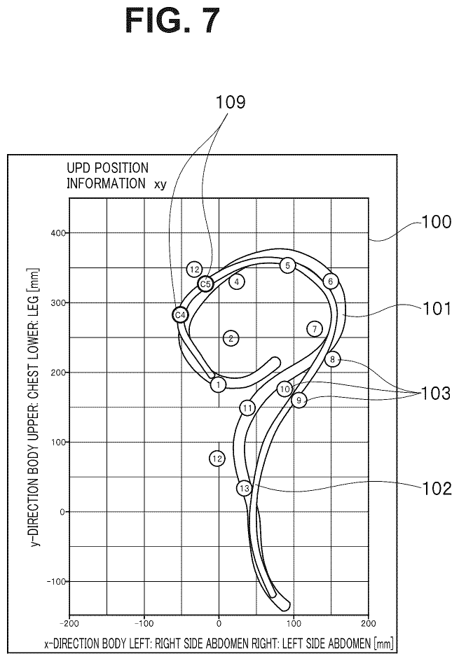

The reference sign 102 in FIG. 4 and FIG. 7 is an example of a graphic showing the position of the distal-end constituting portion 5a and the shape and the arrangement of the insertion portion 5 (hereinafter, referred to as a second insertion shape) corresponding to at least one image acquisition timing among the plurality of endoscopic image acquisition timings. There is one display data for each image acquisition timing. Therefore, when a plurality of images are acquired in one examination, graphic data of the plurality of second insertion shapes are included.

When the distal-end constituting portion 5a detects the plurality of second insertion shapes, the subject (patient) is not necessarily in the supine posture, but may be in a lateral recumbent posture facing sideways. In such a case, the posture of the subject is aligned with the supine posture or a predetermined orientation by correction based on the three-dimensional posture information and the like related to the posture of the subject generated by the reference plate 13, which is attached to the abdomen of the subject. Note that regarding the alignment, the position and the posture may be aligned based on a scope shape.

Here, simultaneously displaying the graphic data of all of the second insertion shapes on a display screen makes the screen display complicated. Therefore, for example, the first display example in FIG. 4 shows an example in which one second insertion shape corresponding to one lesion part among the plurality of lesion parts is displayed. Specifically, FIG. 4 illustrates a display example of the second insertion shape corresponding to a fourth image acquisition timing.

The reference sign 103 in FIG. 4 to FIG. 7 is a display example representing each of the plurality of lesion parts respectively corresponding to the plurality of endoscopic image acquisition timings. In the illustrated examples, the position of the lesion part and the like are estimated and displayed, but for simplicity, they may be replaced by the position of the distal end of the insertion portion 5. There is one piece of display data for each image acquisition timing. Therefore, a plurality of pieces of data are included in one examination.

Here, the first display example of FIG. 4 is the point display corresponding to each of the plurality of lesion parts obtained in one examination. In this case, the number (1 to 13) attached to each point display represents a unique number assigned to each of the plurality of lesion parts.

Note that the number of the image acquisition timings and the number of the lesion parts to be confirmed may increase or decrease depending on the course of the disease for each of the plurality of endoscopies. Therefore, the unique number assigned to each of the plurality of lesion parts may not necessarily be the same for each of the plurality of endoscopies.

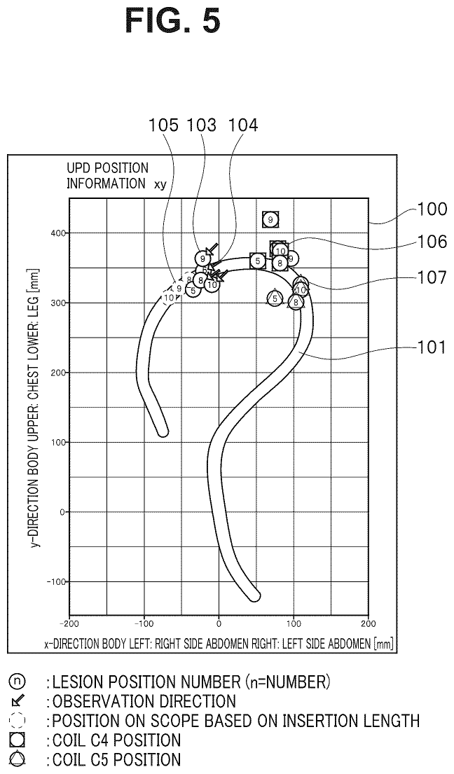

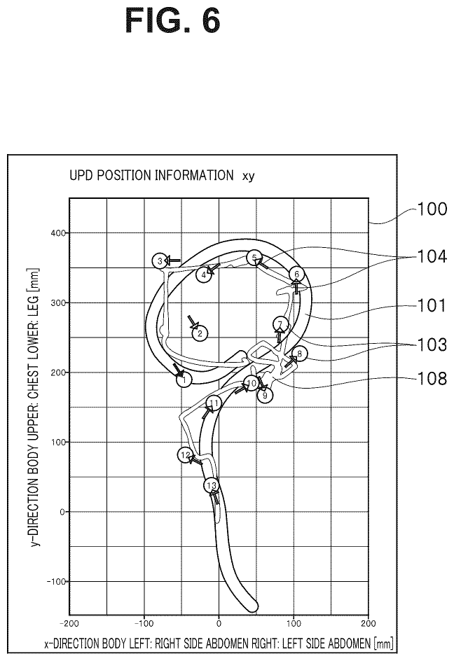

The reference sign 104 in FIG. 5 and FIG. 6 is an arrow display indicating an observation direction corresponding to the position of each lesion part and the like, in addition to the above-described point display 103.

The reference sign 105 in the second display example of FIG. 5 is an example of displaying the position of each lesion part at a corresponding position on the first insertion shape of the insertion portion 5 based on the insertion length information.

The reference sign 106 (indicated by a square mark) in FIG. 5 is an example displaying a specific position (a position of a specific magnetic coil C4, for example) on the insertion portion 5 corresponding to the point display 103 of each of target positions.

The reference sign 107 (indicated by a triangle mark) in FIG. 5 is an example displaying a specific position (a position of a specific magnetic coil C5, for example) on the insertion portion 5 corresponding to the point display 103 of each of the target positions.

Note that displays of the reference signs 103 to 107 are not limited to a form in which all are displayed, but a form in which appropriate displays are combined and displayed may be selected.

The reference sign 108 in the third display example of FIG. 6 is an example of a graphic indicating a movement trajectory of the distal-end constituting portion 5a when the insertion portion 5 is removed. There is one display data for each endoscopy.

The reference sign 109 in the fourth display example of FIG. 7 is an example of the point display representing the position of a specific point (a point corresponding to a specific magnetic coil, for example) on the insertion portion 5. There may be at least one display data. Note that the fourth display example of FIG. 7 shows an example in which two points respectively corresponding to the magnetic coils C4 and C5 (see FIG. 3) are displayed. In addition, the notation (C4, C5) added in the point display is an example of symbols identifying the coil number.

Here, the reason for using the specific points on the insertion portion 5, such as the magnetic coils C4 and C5 is described. Since the bending portion 5b is deformed by the operator, the approach method for lesions located in the unfixed lumen part of the large intestine, such as the sigmoid colon and the transverse colon, is likely to differ, and it is difficult to obtain reproducibility of the positions. In addition, the position of the point on the insertion portion 5 located at the unfixed lumen part of the large intestine, such as the sigmoid colon and the transverse colon tends to vary greatly. Therefore, by selecting a specific point on the insertion portion 5 that is not located in such parts, the reproducibility of the position can be improved.

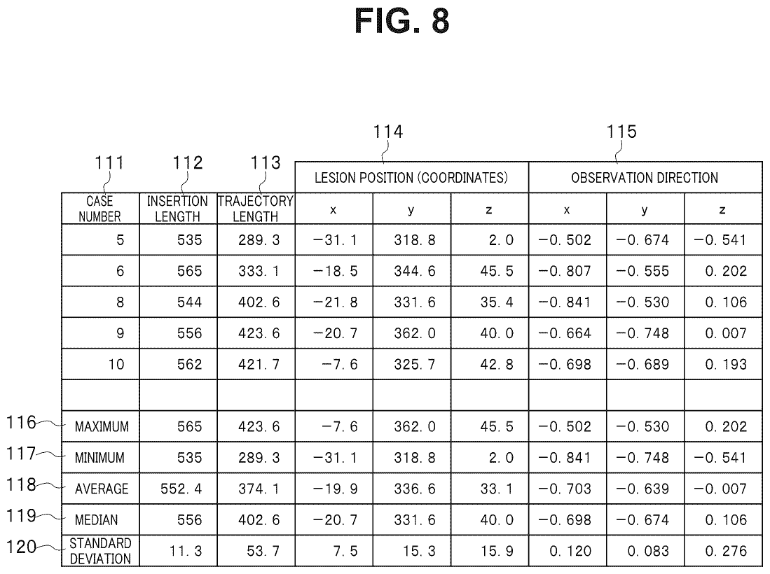

FIG. 8 is a display example of a quantitative display form of the lesion position related information. The quantitative display form of the lesion position related information is, for example, a numerical value table form displaying such that numerical values for each of predetermined items can be compared for each of a plurality of examinations. The predetermined items in this case include, for example, a case number (the reference sign 111), an insertion length from the anus (the reference sign 112), the trajectory length from the intestinal cecum (the reference sign 113), the position coordinates (x, y, z) of the lesion part (the reference sign 114), the vector (x, y, z) indicating the observation direction (the reference sign 115), and the like.

Note that the observation direction of the lesion part and the like coincides with the direction of the distal end of the insertion portion 5, and therefore may be recited as an insertion portion distal end direction.

In addition, when items related to the plurality (three or more) of the lesion parts are displayed side by side, as shown in FIG. 8, indications such as a maximum value (the reference sign 116), a minimum value (the reference sign 117), an average value (the reference sign 118), a median value (the reference sign 119), and a standard deviation (the reference sign 120) may be added, for example.

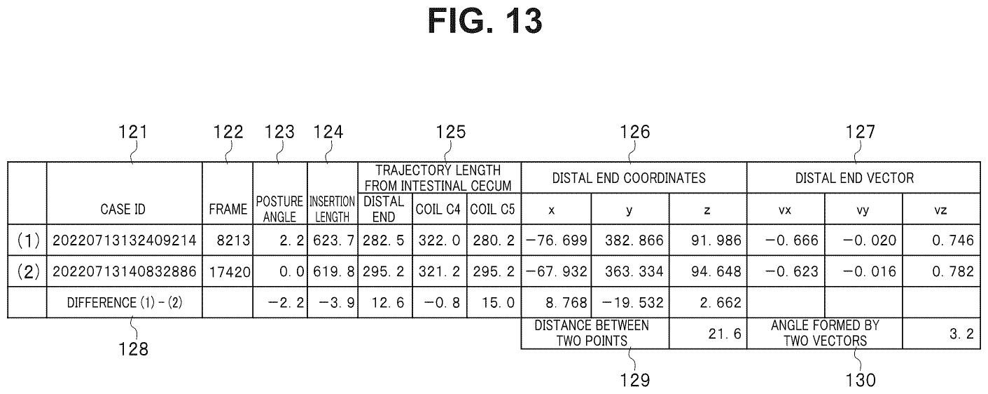

Note that when items related to the plurality (two) of the lesion parts are displayed side by side, a difference may be obtained, and an indication thereof may be displayed (a display form described later: see FIG. 13).

Other than the above, as the lesion position related information, information in association with the endoscopy information and the position information of the corresponding lesion part and the like may also displayed. For example, in addition to the above qualitative display or the quantitative display, the endoscopic image corresponding to the position information of the lesion part and the like may be displayed simultaneously on the display screen.

Next, the lesion position comparison information detection unit 24 in FIG. 2 performs predetermined information processing based on the lesion position related information inputted from the lesion position related information detection unit 23, or a plurality of pieces of predetermined lesion position related information and the like read out from the memory 27 through the storage control unit 26, and detects and generates comparison information of the position in the body of a predetermined lesion part among the plurality of lesion parts and the like in the plurality of endoscopies (hereinafter, referred to as lesion position comparison information).

The lesion position comparison information is generated by performing comparison processing on the plurality of pieces of lesion position related information acquired by the plurality of endoscopies on the same subject. Here, the plurality of pieces of lesion position related information subjected to the comparison processing include, for example, the lesion position related information (output information from the lesion position related information detection unit 23) that is acquired in an endoscopy currently performed, or one or a plurality of pieces of lesion position related information among the lesion position related information acquired in past endoscopies performed in the past on the same subject and stored in the memory 27.

Therefore, the comparison processing between the output information from the lesion position related information detection unit 23 and the information stored in the past may be performed, or the comparison processing among the plurality of pieces of lesion position related information on the same subject stored in the memory 27 may be performed, for example.

Similar to the lesion position related information as described above, the lesion position comparison information is displayed using the display device 29 and the like in a predetermined form such as a qualitative (graphical display, etc.) form or a quantitative (numerical display) form, or in a combination of the both forms, for example.

Furthermore, the lesion position comparison information detection unit 24 has a function of assisting in identification determination to determine whether the lesion part confirmed in the current endoscopy is the same as the lesion part recognized in the past endoscopy, based on the generated lesion position comparison information.

Therefore, the lesion position comparison information detection unit 24 outputs to the display control unit 25 necessary data used for the identification determination processing among the lesion position comparison information. In addition, a result of the identification determination processing is included in the lesion position comparison information and converted into a file, and is outputted and stored in the memory 27 through the storage control unit 26.

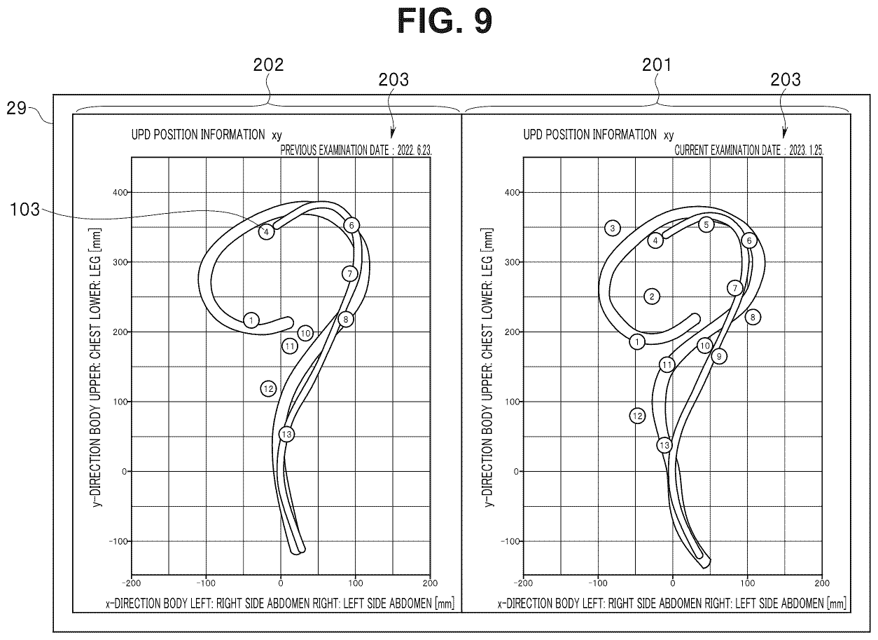

Here, FIG. 9 to FIG. 14 are first to sixth display examples of the lesion position comparison information. First, the first display example of FIG. 9 displays the lesion position comparison information using the qualitative display of the lesion position related information shown in FIG. 4.

In the first display example of FIG. 9, the lesion position related information acquired in the current (latest) examination is displayed side by side with the lesion position related information acquired in the previous (past) examination on the same subject for comparison.

As shown in FIG. 9, on the display screen of the display device 29, the qualitative display of the lesion position related information acquired in the current (latest) examination is shown in a region indicated by the reference sign 201. On the other hand, the qualitative display of the lesion position related information acquired in the previous (past) examination on the same subject is shown in a region indicated by the reference sign 202.

In the first display example of FIG. 9, among the plurality of endoscopies performed on the same subject, the latest information and the past information are displayed in the same display form, and the both are displayed side by side on the same display screen. In addition, the point display (the reference sign 103) attached to the number indicates the target position. Furthermore, respective regions of the reference signs 201 and 202 display examination date information 203, for example. This display form makes it easy to compare two pieces of information, i.e., the latest and past information.

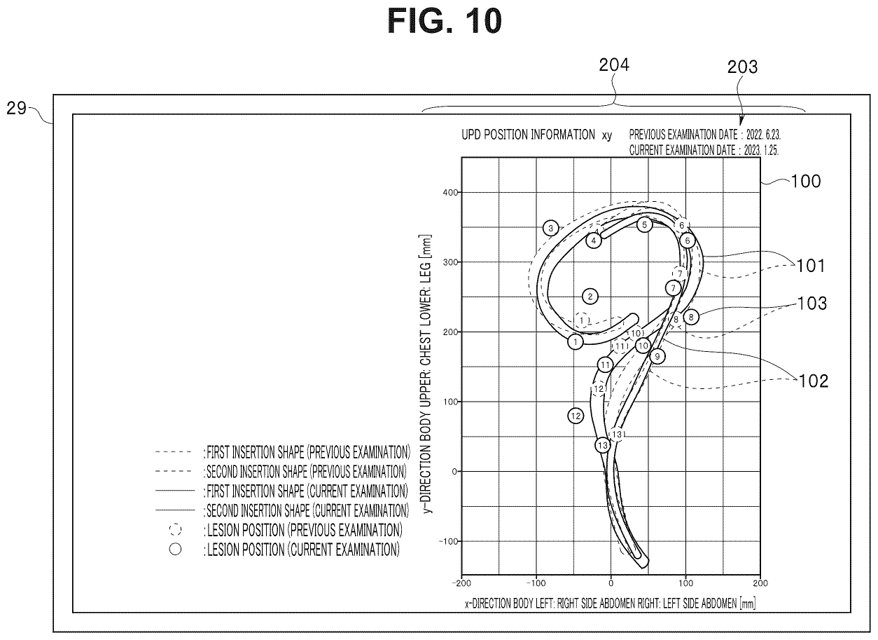

The second display example of FIG. 10 displays the lesion position comparison information using the qualitative display of the lesion position related information shown in FIG. 4. The second display example of FIG. 10 is a display example in which the lesion position related information acquired in the current (latest) examination and the lesion position related information acquired in the previous (past) examination on the same subject are superimposed on the same graph.

As shown in FIG. 10, on the display screen of the display device 29, the qualitative display (solid line display) of the lesion position related information acquired in the current (latest) examination and the qualitative display (dashed line display) of the lesion position related information acquired in the previous (past) examination on the same subject are superimposed in a region indicated by the reference sign 204.

In this case, the qualitative display of the lesion position related information acquired in the current (latest) examination is displayed using the solid line. In addition, the qualitative display of the lesion position related information acquired in the previous (past) examination on the same subject is displayed using the dashed line. Among these, the reference sign 101 indicates the graphic of the first insertion shape. In addition, the reference sign 102 indicates the second insertion shape. The other reference signs are the same as those in the above-described FIG. 4, FIG. 9, and the like. This display form also makes it easy to compare two pieces of information, i.e., the latest information and the past information.

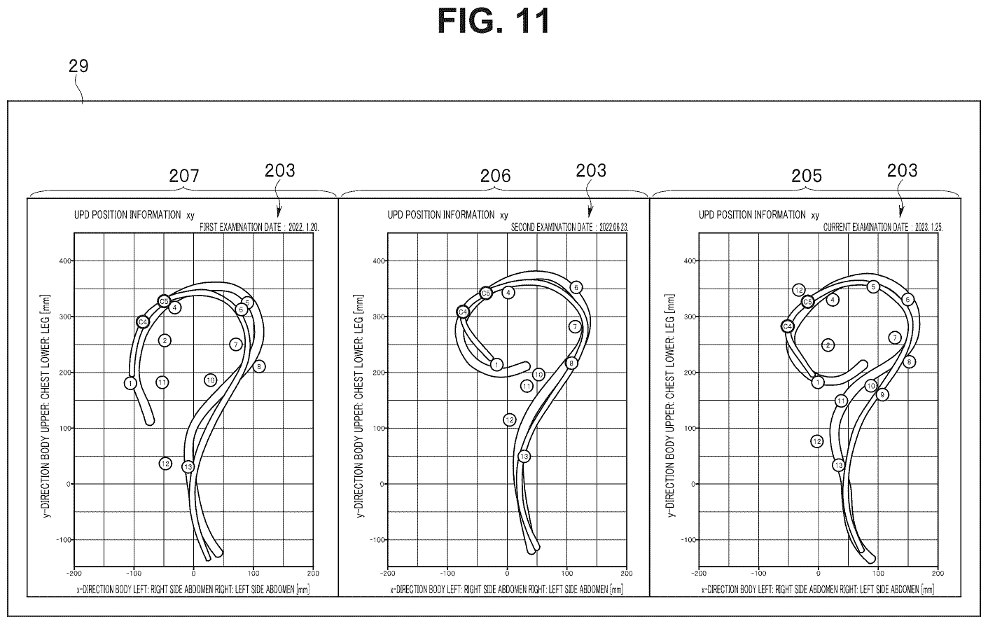

The third display example of FIG. 11 displays the lesion position comparison information using the qualitative display of the lesion position related information shown in FIG. 7. The third display example of FIG. 11 displays the lesion position related information acquired in the plurality (three in the example) times of examinations on the same subject side by side for comparison.

As shown in FIG. 11, on the display screen of the display device 29, the qualitative display of the lesion position related information acquired in the current (latest) examination is shown in a region indicated by the reference sign 205. In addition, the qualitative display of the lesion position related information acquired in the previous (past) examination on the same subject is shown in a region indicated by the reference sign 206. The qualitative display of the lesion position related information acquired in before the previous (further past) examination on the same subject is shown in a region indicated by the reference sign 207.

In the third display example of FIG. 11, among the plurality of endoscopies performed on the same subject, the latest information and the information from the previous two endoscopies are displayed in the same display form, and the three pieces of information are displayed side by side on the same display screen. Other items are the same as those in the above FIG. 7, FIG. 9, and the like. Such display forms make it easy to compare the three, that is, the latest and the two previous examination information and the like.

Note that the third display example of FIG. 11 is an example in which the lesion position related information acquired in the plurality (three) times of examinations are displayed side by side on the same screen, but other display forms different from this example are also possible. For example, two sets of information (the regions indicated by the reference signs 205 and 206, for example) may be displayed side by side on one screen, and by performing a predetermined display switching operation, the display may be instantly switched to other two sets of information (the regions indicated by the reference signs 206 and 207, for example) side by side. Alternatively, one set of information is displayed on one screen, and by any display switching operation, desired information may be displayed.

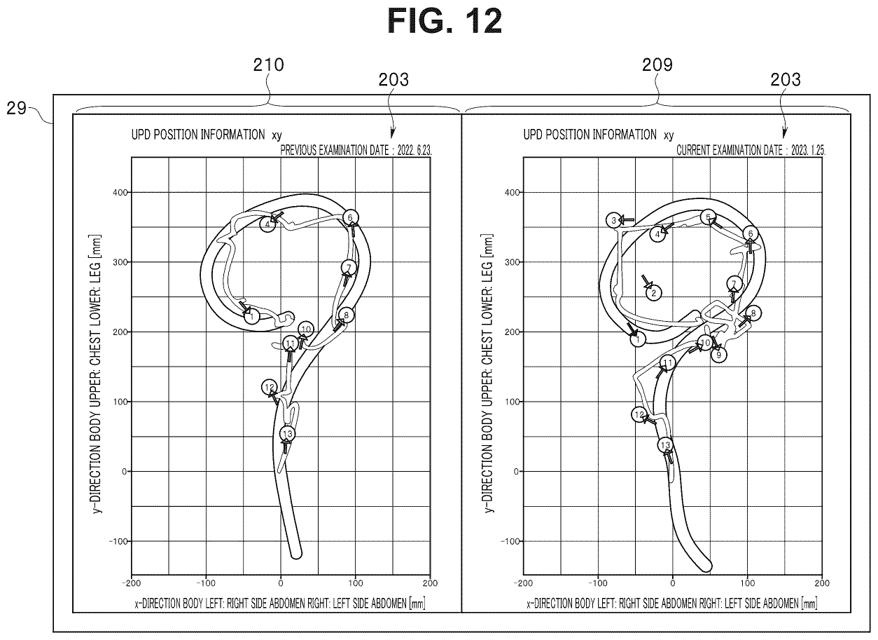

The fourth display example of FIG. 12 displays the lesion position comparison information using the qualitative display of the lesion position related information shown in FIG. 6. The fourth display example of FIG. 12 displays the lesion position related information acquired in the current (latest) examination, and the lesion position related information acquired in the previous (past) examination on the same subject for comparison.

As shown in FIG. 12, the qualitative display of the lesion position related information acquired in the current (latest) examination is shown in a region indicated by the reference sign 209 on the display screen of the display device 29. On the other hand, the qualitative display of the lesion position related information acquired in the previous (past) examination on the same subject is shown in a region indicated by the reference sign 210.

In the fourth display example of FIG. 12, among the plurality of endoscopies performed on the same subject, the latest information and the past information are displayed in the same display form, and the both are displayed side by side on the same display screen. The other items are the same as those in the above-described FIG. 6, FIG. 9, and the like. Such display form makes it easy to compare two pieces of information, i.e., the latest information and the past information.

Note that the fourth display example of FIG. 12 shows graphics indicating the movement trajectories of the distal-end constituting portion 5a at the time of the removal of the insertion portion 5, but is not limited thereto. For example, graphics may be drawn focusing on the trajectory of a specific point (a point corresponding to a specific magnetic coil, for example) on the insertion portion 5 at the time of the removal. In this case, graphics may also be shown substantially similar to those in FIG. 12.

Furthermore, the fifth display example of FIG. 13 shows the lesion position comparison information using the quantitative display of the lesion position related information. The fifth display example of FIG. 13 is a numerical table form similar to the form of FIG. 8. The predetermined items in this case include, for example, a case ID (the reference sign 121), a frame number (the reference sign 122), a posture angle (the reference sign 123), the insertion length from the anus (the reference sign 124), a trajectory length from the intestinal cecum (the reference sign 125), distal end coordinates (the reference sign 126), a distal end vector (the reference sign 127), and the like.

Note that in FIG. 13, the items related to two lesion parts are displayed side by side. Therefore, in FIG. 13, items such as a difference (the reference sign 128), a distance between two points in the coordinates (the reference sign 129), an angle formed by two vectors (the reference sign 130), and the like are additionally displayed.

Incidentally, in the first to fourth display examples of the lesion position comparison information shown in FIG. 9 to FIG. 12, the position information of each lesion part and the like is displayed using only the xy coordinates in the graph 100. However, the position information of each lesion part and the like includes three-dimensional position information (xyz coordinates). Therefore, a display form that utilizes the three-dimensional position information (xyz coordinates) as the position information of each lesion part and the like is conceivable. Furthermore, a display form that combines and displays the quantitative displays of the lesion position related information is also conceivable.

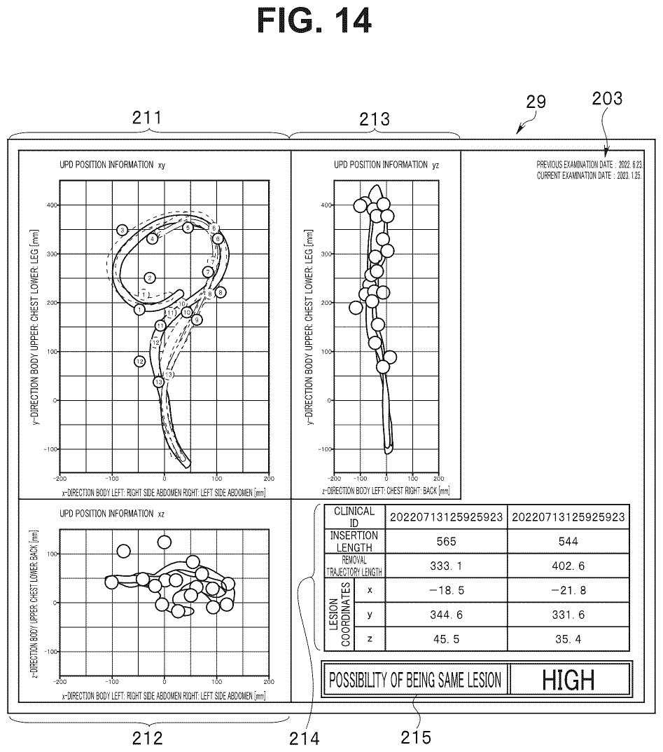

The sixth display example of FIG. 14 is an example of displaying the lesion position comparison information by combining the qualitative display and the quantitative display of the lesion position related information. As shown in FIG. 14, a graph of the xy coordinates indicating the position of each lesion part and the like is displayed in a region indicated by the reference sign 211 on the display screen of the display device 29. The graph display of the xy coordinates is similar to the graph 100 in the display form shown in the second display example of FIG. 10 (the example of superimposed display of the previous examination result and the current examination result).

A graph of the xz coordinates indicating the position of each lesion part and the like is displayed in a region indicated by the reference sign 212. A graph of the yz coordinates indicating the position of each lesion part and the like is displayed in a region indicated by the reference sign 213. Note that in FIG. 14, the specific display in the regions 212 and 213 is simplified to avoid complicating the drawing, but in actuality, the display is performed in accordance with the data.

In a region indicated by the reference sign 214 of FIG. 14, the quantitative display of the lesion position related information for the lesion and the like of the numbered point display 4 is displayed for comparison. In a region indicated by the reference sign 215, a result of the identification determination for the predetermined lesion part is displayed. Here, as shown in FIG. 14, displaying the result of the identification determination includes, for example, an item indicating “POSSIBILITY OF BEING SAME LESION”, and a symbol indicating the identification result in stages, such as “HIGH”, for example.

As a determination criterion, comparative items such as a difference in the coordinates of the lesion and the like, a difference in the insertion length, and an angle formed by orientations of the distal end of the insertion portion 5 (angle difference) are compared with predetermined values respectively, and a comprehensive evaluation is made. For example, examples of the determination criterion includes cases where the angle formed by orientations of the distal end of the insertion portion 5 is 20° or less, and the difference in the position of the lesion and the like is 40 mm or less. By preparing a plurality of predetermined values for each item to be compared, it becomes easier to indicate a matching degree of the comparison result in a multi-stage manner.

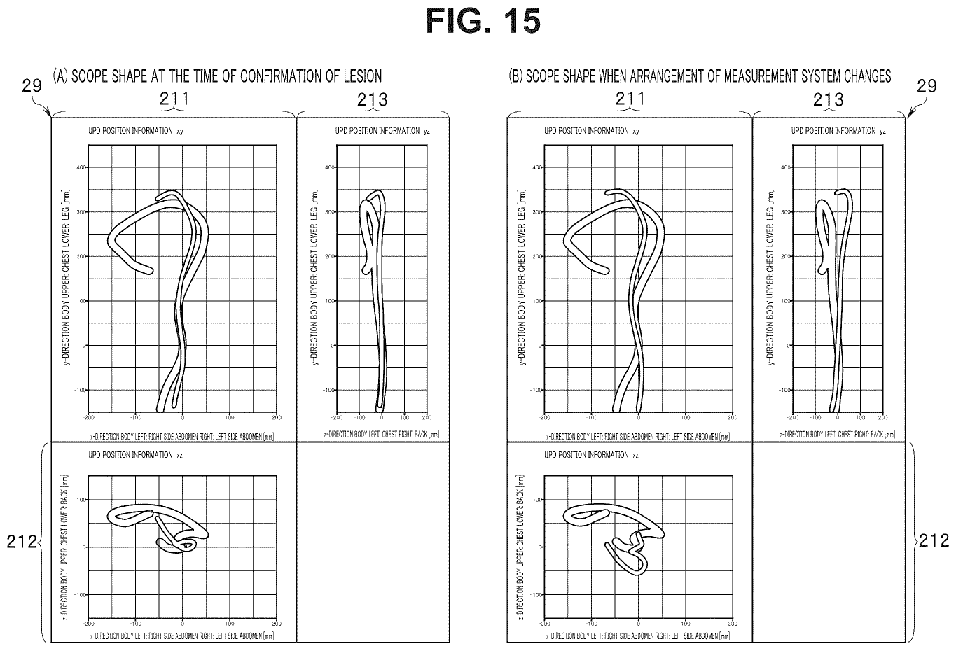

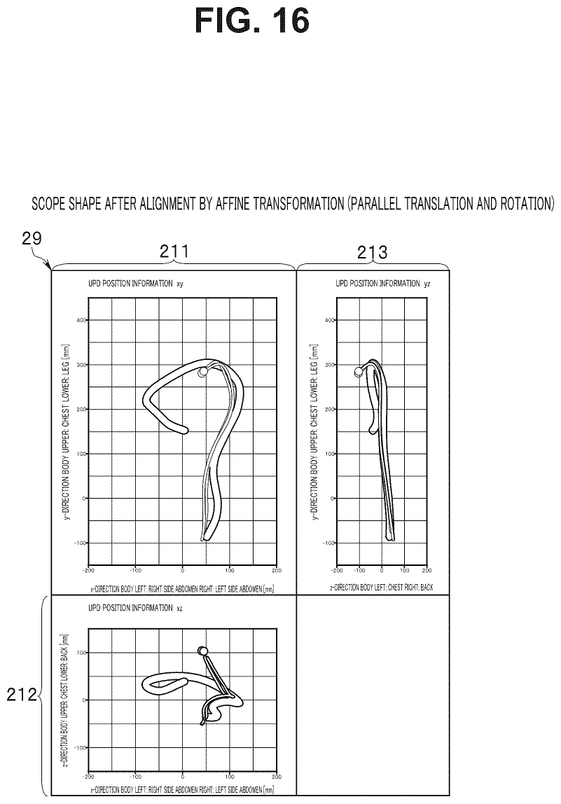

The seventh display example of FIG. 16 displays the lesion position comparison information based on the qualitative display of two pieces of the lesion position related information shown in FIG. 15, in a graph form. Specifically, a graph of the seventh display example of FIG. 16 is a graph created based on alignment of the coordinate systems of the graphs showing spatial arrangements of the endoscope at a plurality of timings.

In order to generate the graph display of FIG. 16, first, the graphs of the qualitative displays of two pieces of the lesion position related information shown in FIG. 15 are acquired. In the graph indicated by the reference sign (A) of FIG. 15, the insertion portion shape at the time of lesion confirmation is displayed in substantially the same form as that in FIG. 14. In the graph indicated by the reference sign (B) of FIG. 15, the insertion portion shape when the arrangement of a measurement system is changed is displayed in substantially the same form as that in FIG. 14.

Here, the respective reference signs shown in FIG. 15 and FIG. 16 are substantially the same as those in FIG. 14. That is, in FIG. 15 and FIG. 16, the reference sign 29 indicates the display screen of the display device 29. In the same figures, a region indicated by the reference sign 211 displays the shape of the insertion portion in the graph of the xy coordinates. A region indicated by the reference sign 212 displays the shape of the insertion portion in the graph of the xz coordinates. A region indicated by the reference sign 213 displays the shape of the insertion portion in the graph of the yz coordinates.

In order to generate the display shown in FIG. 16, the graphs shown in the two acquired data shown in FIG. 15 are used. Here, the data at the plurality of timings for aligning the coordinate systems of the graphs to generate the display in FIG. 16 are two data, i.e., data acquired at the timing indicated by the reference sign (A) of FIG. 15 and data acquired at the timing indicated by the reference sign (B) of FIG. 15, for example.

In addition, the alignment of the coordinate systems of the graphs is performed based on at least the insertion portion shape of the endoscope. That is, the display shown in FIG. 16 is made by the alignment of the coordinate systems of the display data of respective the insertion portion shapes shown by the reference sign (A) and the reference sign (B) of FIG. 15. In this case, for example, parallel translation and rotation (affine transformation) are performed to minimize the error (sum of squares of distances between respective points) at a plurality of points (ten points, for example) at predetermined intervals in an axis direction on the insertion portion (see the reference sign (C) of FIG. 16).

Note that the seventh display example of FIG. 16 is an example of a graph created based on the alignment of the coordinate systems of the graphs, but similarly, it is also possible to generate a comparison table of numerical data indicating the spatial arrangements of the endoscope at the plurality of timings as comparison information based on the alignment of the numerical data of the spatial arrangements.

In addition, lesion identification determination to determine whether lesions are the same may be performed based on images acquired at the plurality of timings, for example.

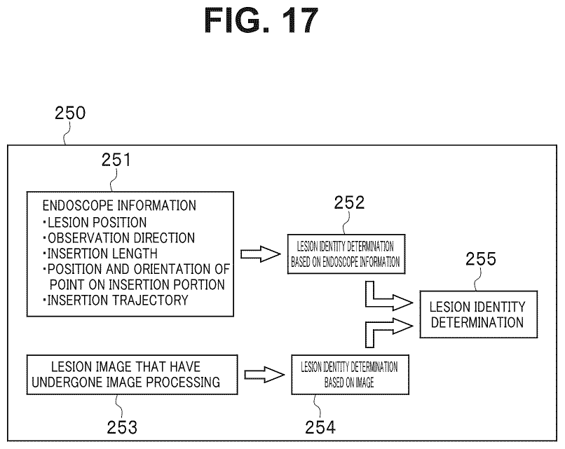

For example, FIG. 17 is a diagram schematically showing an approach for the lesion identification determination based on images. As shown in FIG. 17 and as described above, in the lesion identification determination 250, a lesion identity determination (252) is basically performed based on endoscope information 251 (the lesion position, the observation direction, the insertion length, a position and an orientation of a predetermined point on the insertion portion, the insertion trajectory, and the like). In addition, as shown in FIG. 17, a lesion identity determination (254) is performed by comparing lesion images 253 that have undergone predetermined image processing.

Then, a lesion identity determination 255 is performed by considering both the result of the lesion identity determination (252) based on endoscope information and the result of the lesion identity determination (254) based on the lesion image. In this way, more accurate identity determination can be performed. Note that one or more results derived from the lesion identification determination (including the lesion identity determinations 252, 254, and 255) in this case may also be included in the comparison information. In this case, the comparison information may include at least one of the respective results of the lesion identity determinations 252, 254, and 255. In other words, the comparison information may include not only the result of the lesion identity determination 255 but also the results of the lesion identity determinations 252 and 254. Hereinafter, the lesion identity determinations 252, 254, and 255 may be referred to as a “first lesion identification determination,” a “second lesion identification determination,” and a “third lesion identification determination,”respectively.

Returning to FIG. 2, the display control unit 25 is a configuration circuit or a configuration part that controls the displaying on the display device 29. For example, the display control unit 25 receives various data outputted from the lesion position comparison information detection unit 24, the storage control unit 26, and the like, performs signal processing to generate a predetermined display signal, and outputs the generated display signal to the display device 29. The display device 29 receives the display signal and performs the display in a corresponding form.

The storage control unit 26 is a configuration circuit or a configuration part that receives the lesion position related information detected and generated in the lesion position related information detection unit 23, and the lesion position comparison information detected and generated in the lesion position comparison information detection unit 24, performs predetermined filing processing, and outputs the lesion position related information and the lesion position comparison information to the memory 27. The memory 27 is a storage medium for storing various information and includes a memory region in a predetermined form.

The information (the lesion position related information and the lesion position comparison information) stored in the memory 27 through the storage control unit 26 is outputted to the lesion position comparison information detection unit 24, as necessary. In addition, the information (the lesion position related information and the lesion position comparison information) stored in the storage control unit 26 is outputted to the database device 30 through the communication control unit 28, as necessary.

Furthermore, the information (lesion position related information and the lesion position comparison information) stored in the storage control unit 26 is outputted to the display device 29 through the display control unit 25, as necessary. With this configuration, the display device 29 can receive the information outputted from the display control unit 25 and display various information in the predetermined form.

Note that in the above-described configuration example shown in FIG. 2, an example in which the memory 27 is included inside the endoscope control device 20, but is not limited to such a form. For example, the memory 27 may be configured as an independent and separate storage device outside the endoscope control device 20. In this case, the external storage device is configured to be connected to the storage control unit 26 via a connection cable or wireless communication, for example. Furthermore, the storage control unit 26 and the memory 27 may be configured as an external storage device. In this case, the external storage device can transmit and receive data bidirectionally by being connected to the endoscope control device 20 via a connection cable or the like.

The communication control unit 28 is a configuration circuit or a configuration part including a control unit for performing communication in a predetermined communication form between the endoscope control device 20 and an external device (the database device 30, or the like, for example) to exchange information.

The database device 30 is an external storage device provided in an external device or on the cloud. The database device 30 can exchange information with the endoscope control device 20 of the present embodiment using communication in a predetermined communication form. Note that the database device 30 may includes an external display unit 31, as necessary.

The display device 29 is a display device that is configured independently and separately from a housing of the endoscope control device 20, as shown in FIG. 1 and FIG. 2, for example. Here, as a form of the display device 29, for example, a display device configured using a general liquid crystal panel or the like is applied.

The display device 29 is electrically connected to the endoscope control device 20 via a connection cable or the like. The display device 29 is controlled by the display control unit 25 described later. This enables the display device 29 to receive the display signal outputted from the display control unit 25 and perform various displays. For example, the display device 29 can read out and display the plurality of pieces of lesion position related information and the lesion position comparison information by the past endoscopy stored in the storage control unit 26. In addition, the display device 29 may also display endoscopy information and the like (an endoscopic image and the like, for example) acquired by the endoscope device 1 during the endoscopy.

Note that a configuration example of the display device 29 is not limited to the configuration examples shown in FIG. 1 and FIG. 2. Other configuration examples may include, for example, a form in which the display device 29 is integrated with the housing of the endoscope control device 20.

In addition, the display device 29 does not necessarily have to be a form included in the endoscope control device 20. For example, a form in which the endoscope monitor device 4 included in the endoscope device 1 is used as a display device is conceivable by transmitting the display signal outputted from the display control unit 25 of the endoscope control device 20 to the endoscope device 1 through the connection cable 16.

Furthermore, for example, the display signal outputted from the display control unit 25 of the endoscope control device 20 can be transmitted to the database device 30 through the communication control unit 28. In this case, the database device 30 receiving the display signal can perform predetermined display using the attached external display unit 31.

Note that among the constituent elements of the endoscope control device 20, for example, all or some of the examination information acquisition unit 21, the insertion portion shape and arrangement information acquisition unit 22, the lesion position related information detection unit 23, the lesion position comparison information detection unit 24, the display control unit 25, the storage control unit 26, the memory 27, the communication control unit 28, and the like are configured by an information processing apparatus (a computer) having one or more processors including hardware.

Here, the information processing apparatus is configured by, for example, one or more central processing units (CPUs), a random access memory (RAM), a read only memory (ROM), a non-volatile memory, a non-volatile storage, and the like, as well as a well-known configuration including a non-transitory computer readable recording medium or the like, and peripheral devices thereof. These constituent elements are hardware. A CPU is an example of a processor.

The ROM, the non-volatile memory, the non-volatile storage, and the like store a software program that is executed by the CPUs or fixed data such as a data table, and the like in advance. Then, the CPUs read out the software program stored in the ROM or the like, load the software program into the RAM, and execute the software program, and the CPUs executing the software program refer to various data as appropriate, to realize respective functions of the above configuration circuits or configuration parts (21, 22, 23, 24, 25, 26, and 28) and the like. In this case, the CPUs operate as the above configuration circuits or configuration parts.

In addition, the processor may also be configured using a semiconductor chip or the like such as a field programmable gate array (FPGA). Furthermore, the above configuration circuits or configuration parts (21, 22, 23, 24, 25, 26, and 28) or the like may be configured using an electronic circuit.

Furthermore, the software program may be in a form of a computer program product recorded entirely or partly on a portable plate medium such as a flexible disk, a compact disc read only memory (CD-ROM), a digital versatile disc read only memory (DVD-ROM), or the like, or on a non-transitory computer readable recording medium such as a card-type memory, a hard disk drive (HDD) device, or a solid state drive (SSD) device.

Note that the above-described endoscope system 50 of the embodiment basically includes the endoscope device 1, the UPD 10, the endoscope control device 20, the display device 29, and the database device 30 as in the configuration example shown in FIG. 1 and FIG. 2, but is not necessarily limited to this configuration example.

For example, in the above-described embodiment, the endoscope control device 20 is configured as a dedicated device, but may be configured in various forms as shown below.

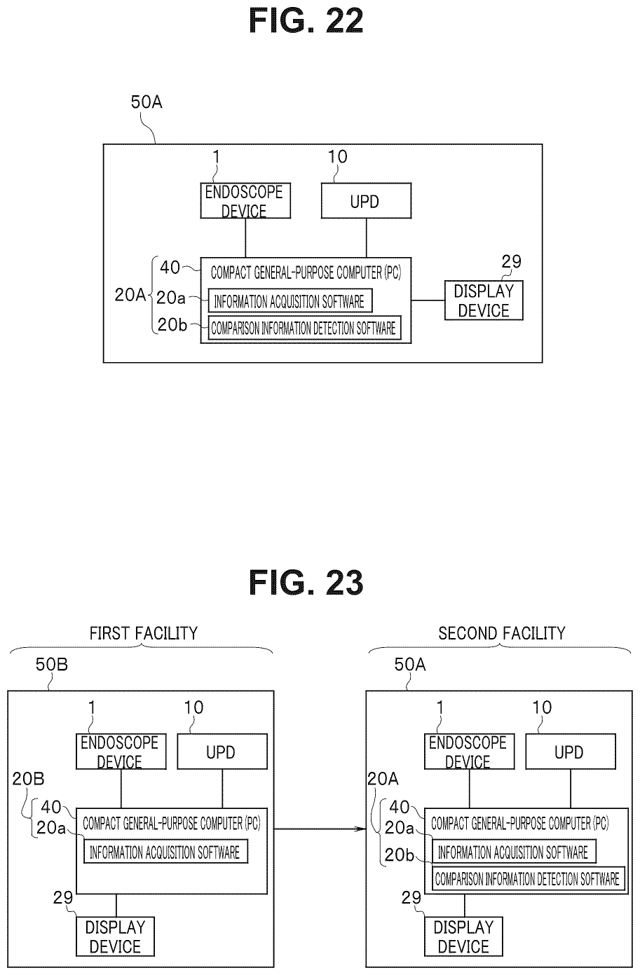

FIG. 22 shows a configuration of a first modification example of the above embodiment. As shown in FIG. 22, an endoscope control device 20A in an endoscope system 50A includes a compact general-purpose computer 40 introducing information acquisition software 20a and comparison information detection software 20b.

Here, the information acquisition software 20a is software that causes the compact general-purpose computer 40 to realize functions of the examination information acquisition unit 21, the insertion portion shape and arrangement information acquisition unit 22, and the lesion position related information detection unit 23 of the endoscope control device 20 of the above-described embodiment.

In addition, the comparison information detection software 20b is software that causes the compact general-purpose computer 40 to realize a function of the lesion position comparison information detection unit 24 of the endoscope control device 20 of the above-described embodiment.

In this configuration, the storage device including the storage control unit 26 and the memory 27 may be in a form (the example as shown) incorporated in the compact general-purpose computer 40, or may be in a form (not shown) of a separate storage device independent of the compact general-purpose computer 40.

In addition, the display device 29 may be a general display device (the example as shown) in an independent and separate form connected to the compact general-purpose computer 40, or may be in a form (not shown) in which the endoscope monitor device 4 (see FIG. 1) provided in the endoscope device 1 is used as a display device.

As a second modification example, a utilization form is assumed in which the same type of endoscopies on the same subject (patient) are performed in a plurality of facilities at different time, and then examination result information acquired in respective facilities are shared and compared.

As a configuration example in this case, for example, as shown in the second modification example of FIG. 23, an endoscope control device 20B of an endoscope system 50B in a first facility utilizes the compact general-purpose computer 40 including at least the information acquisition software 20a. The form of the endoscope system in the first facility is not limited to this form, but may be the configuration of the above-described embodiment, or may be the configuration of the above-described first modification example.

In the first facility, the result information of each endoscopy is stored in a storage device (not shown). The storage device is in a form in which a recording medium is portable. Alternatively, the endoscope control device 20B can transfer data to the endoscope system 50A installed in a second facility, using data communication. In this case, for example, data may be transferred via the database device 30 and the like.

On the other hand, the endoscope control device 20A in the second facility has the same form as that shown in the first modification example described above. The form of the endoscope system in the second facility is also not limited to this form, but may be the configuration of the above-described embodiment.

In the second facility, the result information of each endoscopy is stored in a storage device (not shown). Concurrently, the information of the first facility can be acquired via a storage medium or data communication. This enables the endoscope system 50A in the second facility to compare information data of the examination results transmitted from the first facility with the endoscopy results performed and acquired in the second facility.

Note that, although not shown in the drawings, in a third modification example, endoscopies on the same subject can be performed in a plurality of facilities (examination dedicated facilities, for example). Then, a utilization form is conceivable in which a plurality of examination result information data acquired in the plurality of examination dedicated facilities are transferred to another facility (a research facility, for example), and comparison of data is performed.

As a configuration example in this case, in each of the plurality of examination dedicated facilities, for example, an examination is performed using the endoscope system 50B including the endoscope control device 20B in the form shown in the second modification example described above. Devices used for examination may be the endoscope system 50A including the endoscope control device 20A.

On the other hand, in one research facility, data comparison is performed using the endoscope control device 20 shown in the first and second modification examples described above. The comparison information thus acquired is shared among the plurality of examination dedicated facilities. In this way, comparison of the plurality of endoscopy results acquired in each of the examination dedicated facilities can be performed in the one research facility. In addition, the acquired comparison information can be shared among the endoscope systems in all of the facilities.

Note that the second and third modification examples show the examples of the utilization form in which it is assumed that data is shared among the endoscope systems 50A and 50B respectively installed in two or more facilities, but it is not limited to these examples.

For example, large hospitals may include a plurality of examination systems of the same type. In this case, a configuration including one endoscope system 50A and the plurality of endoscope systems 50B in one facility may be considered.

In this configuration, information data acquired in the plurality of endoscope systems 50B respectively are shared with the endoscope system 50A through an in-hospital server or the like. Therefore, even in this utilization form, comparison of the endoscopy results can be performed in the same manner.

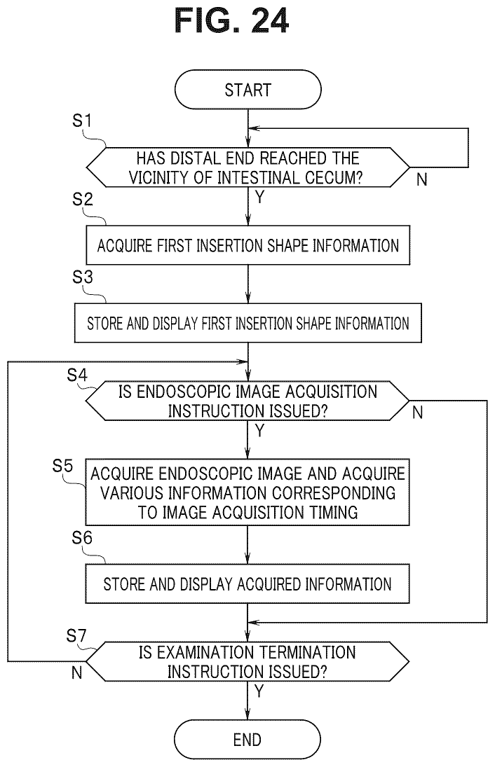

The operation of the endoscope system of the present embodiment configured in this way will be briefly described below. FIG. 24 to FIG. 26 are flowcharts each showing the operation of the endoscope system of the embodiment of the present disclosure. First, FIG. 24 is the flowchart showing the operation when acquiring the examination information, the insertion portion shape and arrangement information and the like during a colonoscopy performed using the endoscope system of the embodiment of the present disclosure. Note that the flowchart of FIG. 24 is the operation of the compact general-purpose computer 40 that executes the information acquisition software 20a.

The endoscope system 50 of the present embodiment is used when performing the colonoscopy on a predetermined subject in a predetermined facility (hospital or the like). In this case, first, the operation when performing the colonoscopy and acquiring the examination information, the insertion portion shape and arrangement information and the like will be described below using FIG. 24.

First, the constituent devices of the endoscope system 50, that is, the endoscope device 1, the UPD 10, the endoscope control device 20, the display device 29, and the like are each set to a usable state. The settings made here include, for example, input setting of individual information (information that can identify the subject, such as a patient number and medical record information) and the like of the subject, which is an object person of the colonoscopy to be performed.

At this time, the subject (patient) is placed in a lateral recumbent posture on an examination table (not shown). Note that the subject may change its posture from the lateral recumbent posture to the supine posture during the examination.

When completing a predetermined preparation of the examination in this way, an operator begins a normal colonoscopy according to a normal procedure (START in FIG. 24).

Here, the operator or the like inserts the insertion portion 5 of the endoscope 2 into the intestinal tract of the large intestine from the anus of the subject. At this time, the operator performs an insertion operation including predetermined operations such as performing a predetermined bending operation to actively bend the bending portion 5b of the insertion portion 5 and applying a twisting operation to the insertion portion 5.

During the insertion operation of the insertion portion 5, the endoscope monitor device 4 displays the endoscopic images. Simultaneously, the display device 29 displays a display based on the insertion shape and arrangement information of the insertion portion 5 acquired by the UPD 10.

In this case, the operator can observe inside the intestinal tract of the large intestine through the endoscopic images during the insertion operation of the insertion portion 5. Therefore, at this time (during the insertion operation), the operator may be able to confirm the lesion part and the like in rare cases.

However, in general, the observation and the examination inside the intestinal tract in the colonoscopy is performed after a distal end part of the insertion portion 5 has reached a predetermined site inside the intestinal tract of the large intestine (usually in the vicinity of the intestinal cecum, which is the deepest portion of the large intestine), while performing a removal operation.

Therefore, while performing the insertion operation of the insertion portion 5, the operator first confirms whether the distal-end constituting portion 5a of the insertion portion 5 has reached the vicinity of the intestinal cecum, which is the deepest portion of the large intestine of the subject (step S1 in FIG. 24).

This confirmation is performed by the operator or the like, based on the display of the endoscopic images and the insertion shape, for example. In other words, in step S1, the control circuit (video processor 3, and the like) in the endoscope device 1 confirms that the distal-end constituting portion 5a of the insertion portion 5 has reached a predetermined position (the vicinity of the intestinal cecum) by detecting the issuance of a stop signal of the insertion operation or an instruction signal to terminate the insertion operation, or the like. Note that the confirmation that the distal-end constituting portion 5a of the insertion portion 5 has reached the predetermined position (the vicinity of the intestinal cecum) may be automatically detected using a predetermined sensor or the like.

At this time, when it is confirmed that the distal-end constituting portion 5a of the insertion portion 5 has reached the vicinity of the intestinal cecum, which is the deepest portion of the large intestine of the subject, the operation proceeds to the next step S2. Note that the confirmation processing is repeated until it is confirmed that the distal-end constituting portion 5a of the insertion portion 5 has reached the vicinity of the intestinal cecum.

Next, in step S2, the UPD 10 outputs first insertion shape information. In response to this, the insertion portion shape and arrangement information acquisition unit 22 of the endoscope control device 20 acquires the first insertion shape information.

The first insertion shape information acquired by the insertion portion shape and arrangement information acquisition unit 22 in this way is outputted to the display control unit 25 and the storage control unit 26 through the lesion position related information detection unit 23.

Next, in step S3, the storage control unit 26 of the endoscope control device 20 performs predetermined signal processing on the inputted first insertion shape information, and outputs the first insertion shape information to the memory 27. In this way, the first insertion shape information is stored in the memory 27.

At the same time, the display control unit 25 of the endoscope control device 20 generates display image data in a predetermined form based on the inputted first insertion shape information, and outputs the display image data to the display device 29. In this way, the first insertion shape information is displayed in a visually recognizable state on the display device 29.

Next, in step S4, the control circuit (video processor 3, and the like) of the endoscope device 1 confirms whether an endoscopic image acquisition instruction signal among the operation instruction signals from the operation member 6c of the operation portion 6 of the endoscope 2 is issued. Here, if it is confirmed that the endoscopic image acquisition instruction signal is issued, the operation proceeds to step S5. In addition, if the endoscopic image acquisition instruction signal is not issued, the operation proceeds to step S7.

In step S5, the control circuit (video processor 3 and the like) of the endoscope device 1 performs predetermined control according to the endoscopic image acquisition instruction signal to acquire the examination information including the endoscopic image and the like. The examination information acquisition control performed here is similar to the control performed by a conventional endoscope device in a general form.

The examination information including the endoscopic image and the like, which are acquired by the endoscope device 1 in this way is outputted from the endoscope device 1 to the examination information acquisition unit 21 of the endoscope control device 20. Therefore, the examination information acquisition unit 21 acquires the examination information.

Furthermore, at this time, a part of the examination information (endoscopic image acquisition timing information or the like, for example) is outputted from the examination information acquisition unit 21 to the insertion portion shape and arrangement information acquisition unit 22. In response to this, the insertion portion shape and arrangement information acquisition unit 22 acquires various information corresponding to the endoscopic image acquisition timing information among the insertion shape information inputted from the UPD 10. Here, the acquired various information includes the position coordinates of the lesion part, the observation direction information of the distal-end constituting portion 5a, the second insertion shape, the insertion length from the anus, the trajectory length from the intestinal cecum, and the like, for example.

The various information acquired in this way are outputted to the display control unit 25 and the storage control unit 26 through the lesion position related information detection unit 23.

Then, in step S6, the storage control unit 26 of the endoscope control device 20 performs predetermined signal processing on the inputted various information, and outputs the various information to the memory 27. In this way, the various information are stored in the memory 27.

At the same time, the display control unit 25 of the endoscope control device 20 performs predetermined signal processing based on the inputted various information to generate the display image data, and outputs the display image data to the display device 29. In this way, for example, the second insertion shape information and the information indicating the position of the lesion part among the various information are displayed in a visually recognizable state on the display device 29. After that, the operation proceeds to step S7.

In step S7, the control circuit (video processor 3 and the like) of the endoscope device 1 confirms whether the examination termination instruction signal among the operation instruction signals from the operation member 6c of the operation portion 6 of the endoscope 2 is issued. Here, if the examination termination instruction signal is not confirmed, the operation returns to step S4 described above, and the subsequent processes are repeated. On the other hand, if the issuance of the examination termination instruction signal is confirmed, the series of processes are terminated (END).

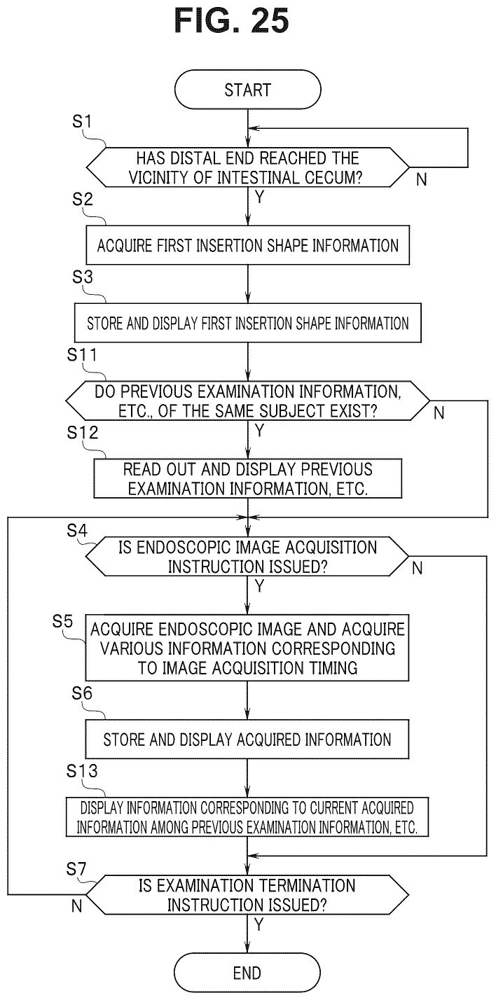

Next, the operation when generating the comparison information based on the various information (the examination information, the insertion portion shape and arrangement information and the like) acquired in each examination in the plurality of colonoscopies will be described below using FIG. 25.

FIG. 25 is a flowchart showing the operation when generating the comparison information based on the plurality of pieces of the examination information and the insertion portion shape and arrangement information acquired for each of the plurality of colonoscopies performed using the endoscope system of the embodiment of the present disclosure. Note that the flowchart of FIG. 25 is the operation of the compact general-purpose computer 40 that executes the comparison information detection software 20b.

Note that, in comparison information processing of the various information acquired by the plurality of colonoscopies, for example, the following cases are considered:
(1) a case where the comparison information is generated based on the various information (real-time information) being acquired by the currently performed colonoscopy and the various information acquired by the previously performed colonoscopy on the same subject (real-time processing); and
(2) a case where the comparison information is generated based on the plurality of pieces of various information acquired by the plurality of colonoscopies previously performed on the same subject (post-processing).

The flowchart of FIG. 25 shows the operation when the above (1) is assumed. Note that a sequence related to the information acquisition processing in the flowchart of FIG. 25 is substantially the same as that of the flowchart of FIG. 24. Therefore, in FIG. 25, processing sequences similar to those of FIG. 24 are attached with the same step numbers, and the detailed descriptions thereof are omitted.

When various settings for each device of the endoscope system 50 are made and the preparation for the examination is completed, the operator starts the normal colonoscopy according to the normal procedure (START of FIG. 25).

The processes in steps S1 to S3 of FIG. 25 are the processing related to the examination information acquisition, and are the same as steps attached with the corresponding same reference signs of FIG. 24.

Next, in step S11, the lesion position related information detection unit 23 of the endoscope control device 20 controls the storage control unit 26 to confirm whether the previous examination information and the previous insertion portion shape and arrangement information (hereinafter, abbreviated as the previous examination information and the like) on the same subject exist in the memory 27.

Here, if it is confirmed that the previous examination information and the like exist, the operation proceeds to the next step S12. If the previous examination information and the like do not exist, for example, after setting a flag indicating that the previous examination information and the like do not exist in the memory 27, the operation proceeds to step S4. In this way, when the operation proceeds to step S4 if the previous examination information and the like do not exist in the memory 27, the processes similar to the processing sequences in FIG. 24 described above are performed.

In step S12, the lesion position related information detection unit 23 controls the storage control unit 26 to read out the previous examination information and the like on the same subject from the memory 27, and transmit the previous examination information and the like to the display control unit 25. In response to this, the display control unit 25 performs predetermined signal processing based on the inputted information to generate the display image data, and outputs the display image data to the display device 29. In this way, necessary information among the previous examination information and the like is displayed in a visually recognizable state on the display device 29.

Here, the memory 27 may store a plurality of pieces of previous examination information and the like on the same subject for the plurality of examinations. Therefore, in the process in step S12, when performing reading processing of the previous examination information and the like into the memory 27, the examination information and the like of the past examination date closest to the current examination date is automatically selected and read.

Furthermore, as another form, for example, the following processing may be further included. In other words, first, in the process in step S12 described above, if it is confirmed that the plurality of pieces of previous examination information and the like on the same subject exist in the memory 27, a list of the previous examination information and the like is displayed on the display device 29. The operator selects and instructs the examination information and the like that the operator wishes to use in the current examination, from the list displayed, using a predetermined input device. Then, based on the selection and instruction signal, the corresponding examination information and the like are read out from the memory 27.

The previous examination information and the like read out from the memory 27 in this way are displayed in a state where the necessary information is visually recognizable as described above. Here, the displayed information includes, for example, the first insertion shape information, the point display representing the plurality of target positions, and the like among the previous examination information and the like that are read.

Thereafter, the processes in steps S4 to S6 are processing related to examination information acquisition, and are the same as steps attached with the corresponding same reference signs of FIG. 24, respectively.

Next, in step S13, the lesion position related information detection unit 23 of the endoscope control device 20 controls the display control unit 25 to perform processing for displaying information corresponding to the current acquisition information among the previous examination information and the like that are read out, on the display device 29. Thereafter, the operation proceeds to step S7. The subsequent processing sequences are similar to those of FIG. 24.

Note that if it is confirmed that in the process in step S4 described above, the previous examination information does not exist (the flag indicating that effect is set), the operation skips step S13 and proceeds step S7.

In addition, when performing the real-time processing, position related information corresponding to the real-time information acquired in the currently performed examination is acquired from the previous examination information and the like read out from the memory 27, the comparison information is generated, and display processing is performed.

In this case, the endoscopic image acquired in the currently performed examination does not necessarily have to show the lesion part and the like. In other words, it is sufficient to compare positional relationships between the position information of the real-time image that is being acquired in the currently performed examination and the position information of the lesion part and the like among the previous examination information and the like, and display the comparison results.

Generated contents of the lesion position related information and the lesion position comparison information in the real-time processing are generally the same. In the real-time processing, the operation information of the endoscope acquired based on the comparison information is further additionally presented. The operation information to be added here includes the following, for example.

For example, there are insertion/removal direction information (when far away), bending operation information (when nearby), an approach method to the lesion part and the like (for observing the lesion part and the like from the front, or observing the lesion part and the like on the underside of a fold), and the like. Note that it is not necessary to provide all endoscope operation information, but information necessary depending on a situation or information that can be guided may be added.

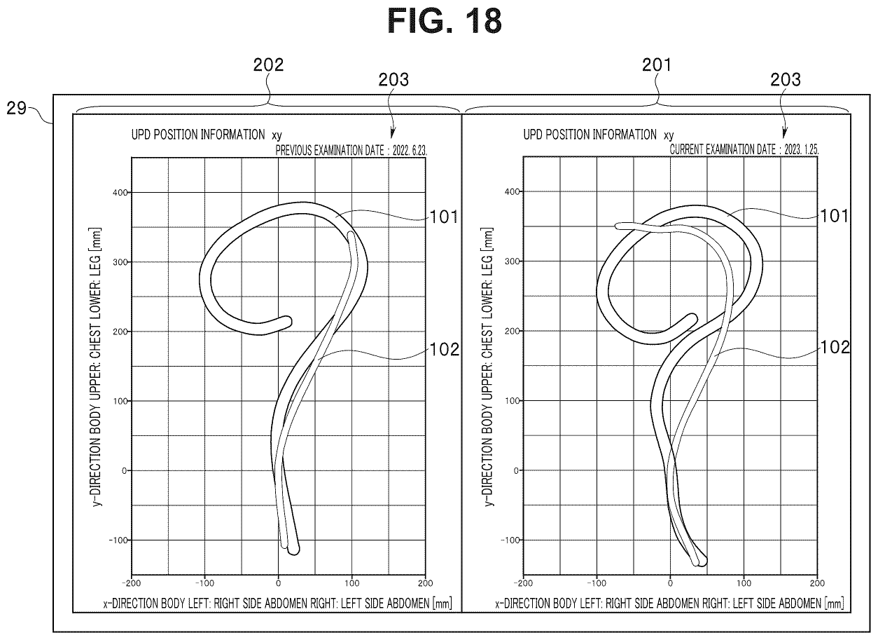

FIG. 18 to FIG. 21 show display examples of the lesion position comparison information during the real-time processing. Note that the respective reference signs shown in FIG. 18 to FIG. 21 are the same as those shown in FIG. 4, FIG. 9, and the like.

FIG. 18 shows an eighth display example of the lesion position comparison information. The example shown in FIG. 18 is an example when a difference between data of the qualitative display 201 of the lesion position related information in the current examination and data of the qualitative display 202 of the lesion position related information in the previous examination on the same subject is large. FIG. 18 shows a case where a difference between the first insertion shape 101 and the second insertion shape 102 is large.

In this case, the operator can bring the distal end portion of the insertion portion closer to a desired lesion and the like by operating the endoscope insertion portion while viewing the difference in the data on the display screen. Therefore, in this case, an operation direction of the insertion portion can be guided by presenting the insertion/removal direction information of the insertion portion.

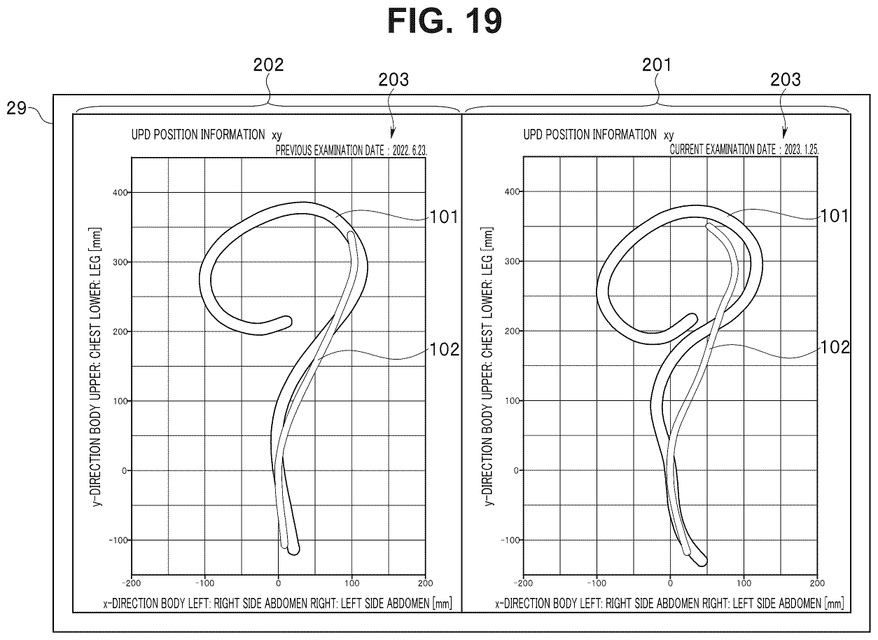

FIG. 19 is a ninth display example of the lesion position comparison information. The example shown in FIG. 19 is an example when a difference between the data of the qualitative display 201 of the lesion position related information in the current examination and the data of the qualitative display 202 of the lesion position related information in the previous examination on the same subject is small. FIG. 19 shows a case where the difference between the first insertion shape 101 and the second insertion shape 102 is small.

In this case, the operator can see from the difference in the data on the display screen that the desired lesion and the like exist in the vicinity of the distal end portion of the insertion portion. Therefore, in this case, the lesion and the like can be easily found by the operation such as slight moving of the orientation and the position of the distal end portion of the insertion portion. Thus, if the bending operation information is presented at this time, the operation direction of the insertion portion can be guided.

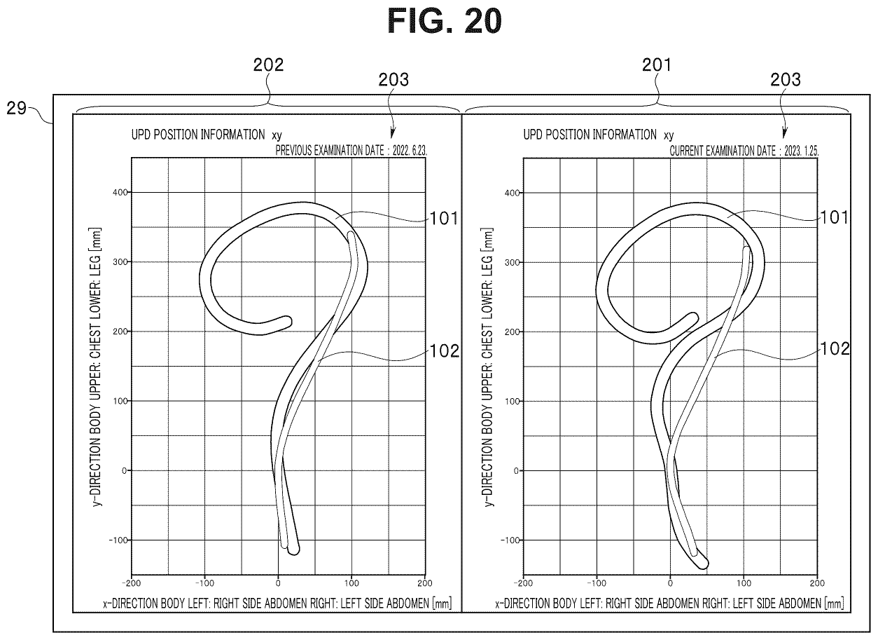

FIG. 20 is a tenth display example of the lesion position comparison information. The example shown in FIG. 20 is an example, in a state of observing the lesion and the like, when the data of the qualitative display 201 of the lesion position related information in the current examination and the data of the qualitative display 202 of the lesion position related information in the previous examination on the same subject are substantially identical. FIG. 20 shows a case where the first insertion shape 101 and the second insertion shape 102 are substantially identical.

In this case, the operator can recognize that the lesion and the like in the displayed endoscopic image is the desired lesion and the like. In this case, in order to further improve accuracy, the identity determination based on image recognition may be performed, for example. This enables reliable identity determination of the lesion and the like.

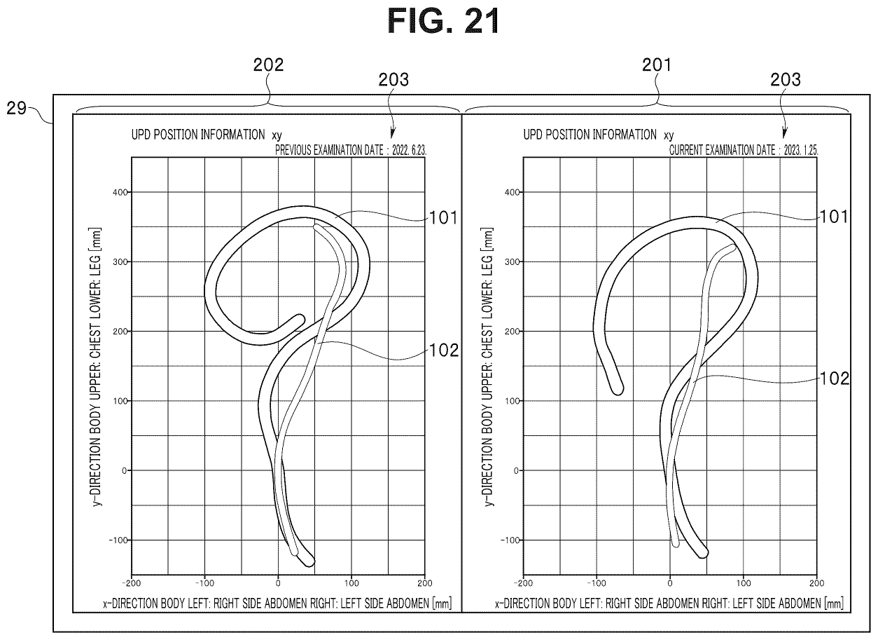

FIG. 21 shows an eleventh display example of the lesion position comparison information. The example shown in FIG. 21 is an example, in a state of observing the lesion and the like, when there is a difference between the data of the qualitative display 201 of the lesion position related information in the current examination and the data of the qualitative display 202 of the lesion position related information in the previous examination on the same subject. FIG. 21 shows a case where there is the difference between the first insertion shape 101 and the second insertion shape 102.

In this case, the operator cannot immediately determine whether the lesion and the like in the displayed endoscopic image is the desired lesion and the like. In this case, a different approach method for the lesion and the like is presented. As the different approach method for the lesion and the like, for example, an approach method for presenting a frontal vision of the lesion part and the like, or for observing the lesion part and the like on the underside of the fold is presented. In addition, in order to further improve the accuracy, the identity determination based on the image recognition may be performed, for example. This enables reliable identity determination of the lesion and the like.

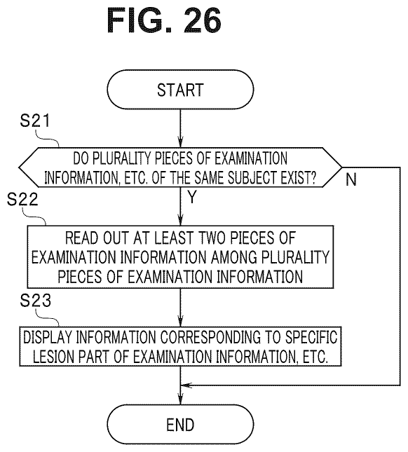

Next, the operation when assuming the above (2) in comparison information generation processing will be described below using the flowchart of FIG. 26.

In this case, the endoscope control device of the endoscope system may include at least the function of the lesion position comparison information detection unit 24 (the function of the comparison information detection software 20b), and the functions of the examination information acquisition unit 21, the insertion portion shape and arrangement information acquisition unit 22, and the lesion position related information detection unit 23 (the function of the information acquisition software 20a) are not required.

First, in step S21, the lesion position related information detection unit 23 of the endoscope control device controls the storage control unit 26 to confirm whether the plurality of pieces of the examination information and the like for the plurality of examinations on the same subject exist in the memory 27. Here, if it is confirmed that the plurality of pieces of examination information and the like for the plurality of examinations exist, the operation proceeds to the next step S22. If the plurality of pieces of examination information and the like do not exist, the series of processes are terminated (END).

Next, in step S22, the lesion position related information detection unit 23 controls the storage control unit 26 to read out the plurality (at least two) pieces of examination information among the plurality of pieces of examination information and the like for the plurality of examinations on the same subject from the memory 27, and transmit the plurality (at least two) pieces of examination information to the display control unit 25.

In the next step S23, the display control unit 25 performs predetermined signal processing based on the inputted plurality of pieces of examination information, and generates and outputs the display image data to the display device 29. Thus, the necessary information among the examination information and the like is displayed in the visually recognizable state on the display device 29.

Here, the display is in a form that allows a user to compare the information corresponding to the predetermined lesion part that the user wishes to perform a comparative observation, among the respective examination information and the like, for example. Specifically, for example, the display is in the form of the display examples of the lesion position comparison information shown in FIG. 9 to FIG. 13 and the like described above.

As described above, according to the above embodiment, when performing the colonoscopy, the examination information and the like are acquired and stored for each examination. Then, when the plurality of colonoscopies on the same subject are performed, the various information regarding the plurality of lesion parts can be provided as the comparison information displayed in the predetermined display form, by using the plurality of pieces of examination information and the like acquired in the plurality of examinations.

The predetermined display form in this case is a form in which the pieces of information are simultaneously displayed side by side on the same screen, a form in which the pieces of information are superimposed and displayed on the same screen at the same scale, a form in which the pieces of information are switched and displayed on the same screen at the same scale, or the like.

By displaying the comparison information in this way, according to the present embodiment, the pieces of information and the like related to the plurality of lesion parts acquired in the colonoscopies on the same subject are compared, thereby assisting in performing more accurate and easier identification determination whether the plurality of lesion parts are the same lesion part.

Therefore, when performing the same type of examination (colonoscopy) again on the same subject, it is possible to easily re-approach the lesion part and the like, which has been confirmed in the previous examination. This makes it possible to easily and quickly perform a follow-up observation of the predetermined lesion part and the like, which has been confirmed. In addition, in a case where a treatment was not performed in the previous examination, it becomes possible to easily identify the lesion part and the like, which is a target, and to quickly perform the necessary treatment.

Note that as the determination criteria of the identification determination of the lesion part, predetermined values based on a distribution of clinical data, a detection accuracy of a detection system, and the like is used. In addition, optimization methods, AI, and the like may be used to select and combine quantitative information items, and to derive determination formulas and coefficients using these items.

In addition, the determination results may be displayed in a digital binary form indicating whether the predetermined lesion part and the like are at the same position or a different position, or in a form indicating the reliability of the determination using numerical values in stages.

Note that the display examples of the determination results include classification items such as “the lesion parts and the like are in the same position”, “the lesion parts and the like are in different positions”, or the like, for example. In addition, the classification items may include subclassifications such as “nearby”, “far away”, or the like, for example.

Furthermore, another display example of the determination results includes items such as “difficult to determine” or “unable to determine”. In this case, subclassifications such as “near but cannot be determined whether in the same position” or “difficult to determine because the approaches of the insertion portion are different” may be further added.

The endoscope device 1 of the present embodiment includes one or more processors including hardware. The one or more processors acquire each endoscope information when the endoscope is located in the observation target region at the plurality of timings, and generate the comparison information regarding the observation target region. With this configuration, when performing the plurality of colonoscopies on the same subject, the endoscope device 1 of the present embodiment can more reliably perform the identification determination whether the lesion and the like confirmed in the currently performed examination and the lesion and the like that have been confirmed in the previously performed examination (including traces or the like after the treatment) are the same lesion and the like.

Incidentally, when one colonoscopy is performed on the same subject, an observation or the like may be performed again on the lesion and the like that have already been confirmed once. For example, the lesion and the like that were confirmed once during the examination may be lost sight of, during the examination. In addition, the lesion and the like found during the insertion operation of the endoscope insertion portion into the intestinal cecum may be confirmed again at the time of the observation or the examination during the removal operation.

In addition, a plurality of image pickup operations may be performed during a detailed examination or differentiation of the lesion and the like, or the image pickup operation may be performed before and after a predetermined treatment. In such cases, a method of the image pickup operation (observation angle, or the like) may change, or a treatment may be performed, making it impossible to immediately determine whether the lesions and the like are the same by simply looking the image pickup results (images).

Therefore, according to the endoscope device 1 of the present embodiment, since the endoscope information is acquired at a plurality of timings during the same examination, it is possible to more reliably perform the identification determination whether the lesions and the like confirmed at the plurality of timings even during the one examination are the same lesion and the like.

Furthermore, in terms of software, it is sufficient to be able to compare the endoscope information acquired at the plurality of timings from the same examination, and even in a configuration with no substantial changes, the function of the identification determination of the lesion and the like can be realized.

The comparison information includes lesion position information in the subject, and information regarding the observation direction (that is, an approach direction of the endoscope insertion portion). By including a plurality of pieces of information as the comparison information in this way, even if there are differences in the position information of the lesion and the like as a result of the comparison, it is possible to confirm the possibility that the lesions and the like are the same if the observation direction information matches. In addition, even if the position information of the lesion and the like generally matches, if the observation direction differs significantly, there is a possibility that the lesions and the like are different.

In this way, by combining and comparing the position information of the lesion and the like and the observation direction information, it is possible to perform a more reliable identification determination whether the lesions and the like are the same.

The comparison information is displayed in the form of a graph showing the spatial arrangements of the endoscope at the plurality of timings. Therefore, by displaying information regarding the position of the lesions and the like on the intestinal tract acquired at two timings in a graph, it is possible to quickly and reliably perform the identification determination whether or not the lesions and the like are the same at a glance.

The information to be displayed in the graph includes information regarding the shape of the insertion portion of the endoscope in the subject. In this way, by displaying the shape of the insertion portion of the endoscope in the graph, the shape of the endoscope insertion portion such as a site where the distal end of the endoscope insertion portion is located, the insertion shape of the insertion portion, the observation direction of the lesion and the like can be generally confirmed. By providing a display form in which the information can be visually confirmed in the graph, it is possible to quickly and more reliably perform the identification determination whether the lesions and the like are the same.

The graph display is created based on the alignment of the coordinate systems of the graphs of the spatial arrangements of the endoscope at the plurality of timings. In this way, by displaying the position information on the intestinal tract related to the lesion and the like on one graph, it is possible to clearly confirm the matching degree and the differences of the position information at the plurality of timings. Therefore, it is possible to quickly and more reliably perform the identification determination whether the lesions and the like are the same.

The coordinate systems of the graphs are aligned based on at least the insertion portion shape of the endoscope. For example, the position and the shape information and the like of the endoscope insertion portion at the plurality of timings may be misaligned due to differences in the arrangement of the subject (patient) on the examination table or settings of the measurement system.

Therefore, by aligning measurement data each obtained under different conditions, such as a difference in cases, a difference in the setting of the measurement system, and a difference in the measurement system, based on the insertion position and the shape of the endoscope insertion portion, comparison with higher accuracy can be made.

Here, as a method of the alignment, for example, in different cases of the same subject, the shapes when the endoscope insertion portion is disposed at the intestinal cecum or the deepest portion of the insertion are compared for each part of the insertion portion. Then, the method of the alignment of the coordinate systems can be determined by parallel translation and rotation of the coordinate system so as to minimize a total error. Note that shapes may be taken at the plurality of timings from two cases and compared.

The comparison information may be configured to include a comparison table of numerical data. The above-described graph display is useful for a doctor, the operator, or the like to quickly perform the identification determination whether the lesions and the like are the same. However, it can be said that the graph display is not suitable for performing a strict comparison.

Then, when performing the strict comparison, a comparison using the numerical data is performed, for example. By doing this, it is possible to confirm items with high or low matching degrees, a level of the matching degrees, and the like. In other words, by displaying the results using the numerical data, it is possible to perform the identification determination whether the lesions and the like are the same with higher accuracy and provide clear grounds for the identification determination.

The endoscope information includes at least one of the insertion length, the insertion trajectory, the lesion position coordinates, the position coordinates of the distal end of the insertion portion, or the lesion observation direction. Each of the information is useful information for identifying the position of the lesion and the like on the intestinal tract. Depending on the insertion state of the endoscope insertion portion at a timing when the lesion and the like are confirmed, the information that is useful when performing the identification determination whether the lesions and the like are the same, with high reproducibility varies.

Among these, the position coordinates and the observation direction are likely to match when the endoscope insertion portion is operated in the same manner at different timings.

In addition, the insertion length is likely to match when observing the same lesion and the like even if the distal end position changes in a case where a path of the insertion portion tends to waver in the sigmoid colon or the transverse colon.

In the sigmoid colon and the transverse colon, the movement of the vicinity of the distal end of the insertion portion may deviate from the insertion portion shape when reaching the intestinal cecum, but the insertion trajectory may be information representing such a tendency.

In this way, by using at least one of the insertion length, the insertion trajectory, the lesion position coordinates, the insertion portion distal end position coordinates (i.e., the coordinates of the distal end position of the insertion portion), or the lesion observation direction as the endoscope information depending on the situation, it is possible to further improve the accuracy when performing the identification determination whether the lesions and the like are the same.

Furthermore, the endoscope information may be a combination of at least two of the insertion length, the insertion trajectory, the lesion position coordinates, the insertion portion distal end position coordinates, or the lesion observation direction.

By using, for example, a plurality (two or more) items among the information simultaneously as the endoscope information for identifying the position of the lesion and the like on the intestinal tract, it is possible to further improve the accuracy when performing the identification determination whether the lesions and the like are the same, or to supplement each other.

For example, when the matching degree between information of the plurality of items is high, the identification determination can be performed with higher accuracy. In addition, when one piece of information does not function effectively, other information may function as identification information. An example of this case may include, for example, a combination of information such as the lesion position coordinates, the lesion observation direction, and the insertion length.

The comparison information includes the determination result of the lesion position identification. For example, if a criterion of the identification determination for the same lesion and the like can be obtained statistically, the determination can be performed using the criterion. Obtaining such the determination results can reduce the efforts of the operator or the like to perform determination based on a vague determination criterion such as experiences and intuitions, while also reducing the possibility of erroneous determination.

The comparison information further includes a lesion identification determination result indicating whether the lesions are the same based on images at the plurality of timings. In general, it is difficult to always obtain 100% accuracy in the determination result of the identification determination for the same lesion and the like obtained using the endoscope information based on the position and the shape of the endoscope insertion portion. Therefore, if the identity can be determined by directly comparing images of the lesions and the like at different timings, the accuracy of the determination can be improved.

Position relationship information between a site currently observed and the observation target region including the lesion and the like that have been recognized in the past is presented. By presenting a relationship between the position of the observation target region of the target lesion and the like and the position of the site currently observed in real time during the colonoscopy, it becomes possible to do the following.
1) If the positional relationship between the site currently observed and the observation target region including the lesion and the like that have been recognized in the past is distant, it is possible to approach the lesion and the like based on the difference in the positions. In this case, the insertion or removal operation may be mainly performed as an operation to approach the lesion and the like.
2) If the positional relationship is close, the vicinity of the site currently observed can be carefully searched because it is known that the lesion and the like are nearby. In this case, the bending operation or the slight insertion or removal operation may be performed.

The operation information is generated from information regarding the site currently observed and the observation target region that has been recognized in the past. In real time during the colonoscopy, in addition to the relationship between the site of the observation target region of the target lesion and the like and the position of the site currently observed, the following operations to bring the insertion portion distal end to the position of the same lesion and the like can be presented.
1) If the positional relationship is distant, an operation to approach the lesion and the like, mainly the insertion or removal operation can be presented.
2) If the positional relationship is close, the bending operation or the twisting operation to align the directions, the insertion or removal operation to slightly adjust the position in the insertion direction, and the like can be presented.

The present disclosure is not limited to the above-described embodiments, and it goes without saying that various modifications and applications can be implemented within a range without departing from the gist of the disclosure.

Furthermore, the above embodiments include disclosures at various stages, and various disclosures can be extracted by appropriate combinations of a plurality of disclosed constituent elements. For example, even if some of the constituent elements are deleted from all the constituent elements shown in the above-described one embodiment, a configuration from which the constituent elements are deleted can be extracted as a disclosure insofar as the configuration can solve the problem to be solved by the disclosure and attain the effects of the disclosure. Furthermore, constituent elements over different embodiments may be appropriately combined. The disclosure is not limited by the specific embodiments except as limited by appended claims.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor. ” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B. ” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be Described In The Following Clauses:
Clause 1: An endoscope control device comprising:
   one or more processors including hardware, wherein
   the one or more processors acquire endoscope information and image information of an observation target region at each of a plurality of timings when an endoscope is located in the observation target region, and generate comparison information regarding the observation target region,
   the comparison information includes lesion identification determination result indicating whether lesions are the same at the plurality of timings, and
   the one or more processors
      perform a first lesion identification determination to determine whether the lesions are the same based on the endoscope information,
      perform a second lesion identification determination to determine whether the lesions are the same based on the image information, and
      determine whether the lesions are the same based on a result of the first lesion identification determination and a result of the second lesion identification determination.
Clause 2: The endoscope control device according to clause 1, wherein each of the plurality of timings is a timing when the endoscope information is acquired in a same examination.
Clause 3: The endoscope control device according to clause 1, wherein the comparison information includes information regarding a lesion position and an observation direction in a subject.
Clause 4: The endoscope control device according to clause 1, wherein the one or more processors output a signal for displaying a graph showing spatial arrangements of the endoscope at the plurality of timings, as the comparison information.
Clause 5: The endoscope control device according to clause 4, wherein information displayed in the graph includes information regarding a lesion position and an observation direction in a subject.
Clause 6: The endoscope control device according to clause 4, wherein information displayed in the graph includes information regarding a shape of an insertion portion of the endoscope in a subject.
Clause 7: The endoscope control device according to clause 4, wherein the graph is created based on alignment of coordinate systems of graphs of the spatial arrangements at the plurality of timings.
Clause 8: The endoscope control device according to clause 7, wherein the alignment of the coordinate systems of the graphs is performed based on at least a shape of an insertion portion of the endoscope.
Clause 9: The endoscope control device according to clause 1, wherein the one or more processors output a signal for displaying a comparison table of numerical data indicating spatial arrangements of the endoscope at the plurality of timings, as the comparison information.
Clause 10: The endoscope control device according to clause 9, wherein the comparison table of the numerical data is created based on alignment of numerical data of the spatial arrangements at the plurality of timings.
Clause 11: The endoscope control device according to clause 10, wherein the alignment of the numerical data is performed based on at least a distance between two predetermined points of an insertion portion of the endoscope.
Clause 12: The endoscope control device according to clause 1, wherein the endoscope information is at least one of an insertion length, an insertion trajectory, lesion position coordinates, insertion portion distal end position coordinates, or a lesion observation direction.
Clause 13: The endoscope control device according to clause 12, wherein the endoscope information is a combination of at least two of the insertion length, the insertion trajectory, the lesion position coordinates, the insertion portion distal end position coordinates, or the lesion observation direction.
Clause 14: The endoscope control device according to clause 1, wherein the first lesion identification determination based on the endoscope information is performed based on an insertion length of an insertion portion of the endoscope, an orientation of a distal end of the insertion portion of the endoscope, and coordinates of a lesion.
Clause 15: The endoscope control device according to clause 1, wherein the one or more processors present positional relationship information between a site currently observed and an observation target region including a lesion and the like that have been recognized in a past.
Clause 16: The endoscope control device according to clause 15, wherein the one or more processors generate operation information from the site currently observed and the observation target region that has been recognized in the past.
Clause 17: An endoscope system comprising:
   an endoscope; and
   a control device including one or more processors including hardware, wherein
   the one or more processors acquire endoscope information and image information of an observation target region at each of a plurality of timings when an endoscope is located in the observation target region, and generate comparison information regarding the observation target region,
   the comparison information includes lesion identification determination result indicating whether lesions are the same at the plurality of timings, and
   the one or more processors
      perform a first lesion identification determination to determine whether the lesions are the same based on the endoscope information,
      perform a second lesion identification determination to determine whether the lesions are the same based on the image information, and
      determine whether the lesions are the same based on a result of the first lesion identification determination and a result of the second lesion identification determination.
Clause 18: An endoscope control method comprising:
   acquiring, by one or more processors including hardware, endoscope information and image information of an observation target region at each of a plurality of timings when an endoscope is located in the observation target region; and
   generating, by the one or more processors, comparison information regarding the observation target region, wherein
   the comparison information includes lesion identification determination result indicating whether lesions are the same at the plurality of timings, and
   the generating the comparison information includes:
      performing a first lesion identification determination to determine whether the lesions are the same based on the endoscope information;
      performing a second lesion identification determination to determine whether the lesions are the same based on the image information; and
      determining whether the lesions are the same based on a result of the first lesion identification determination and a result of the second lesion identification determination.
Clause 19: The endoscope control method according to clause 18, wherein each of the plurality of timings is a timing when the endoscope information is acquired in a same examination.

What is claimed is:

1. An endoscope control device comprising:
a connection interface; and
one or more processors comprising hardware, configured to:
acquire, via the connection interface, endoscope information and image information of an observation target region at a plurality of timings at which an endoscope is positioned in the observation target region; and
perform lesion identification determinations each determining whether lesions observed at the plurality of timings are identical to each other, thereby generating comparison information including one or more results derived from the lesion identification determinations, the lesion identification determinations comprising:
a first lesion identification determination based on the endoscope information;
a second lesion identification determination based on the image information; and
a third lesion identification determination based on results of the first lesion identification determination and the second lesion identification determination.

2. The endoscope control device according to claim 1, wherein the plurality of timings are timings at which the endoscope information is acquired in a same examination.

3. The endoscope control device according to claim 1, wherein the comparison information includes information regarding a lesion position and an observation direction in a subject.

4. The endoscope control device according to claim 1, wherein at least one of the one or more processors is configured to output a signal for displaying, as the comparison information, a graph showing spatial arrangements of the endoscope at the plurality of timings.

5. The endoscope control device according to claim 4, wherein information displayed in the graph includes information regarding a lesion position and an observation direction in a subject.

6. The endoscope control device according to claim 4, wherein information displayed in the graph includes information regarding a shape of an insertion portion of the endoscope in a subject.

7. The endoscope control device according to claim 4, wherein the graph is created based on alignment of coordinate systems of graphs showing the spatial arrangements of the endoscope at the plurality of timings.

8. The endoscope control device according to claim 7, wherein the alignment of the coordinate systems of the graphs is performed based on at least a shape of an insertion portion of the endoscope.

9. The endoscope control device according to claim 1, wherein at least one of the one or more processors is configured to output a signal for displaying, as the comparison information, a comparison table of numerical data indicating spatial arrangements of the endoscope at the plurality of timings.

10. The endoscope control device according to claim 9, wherein the comparison table is created based on alignment of numerical data of the spatial arrangements of the endoscope at the plurality of timings.

11. The endoscope control device according to claim 10, wherein the alignment of the numerical data is performed based on at least a distance between two predetermined points of an insertion portion of the endoscope.

12. The endoscope control device according to claim 1, wherein the endoscope information includes at least one of an insertion length, an insertion trajectory, lesion position coordinates, insertion portion distal end position coordinates, or a lesion observation direction.

13. The endoscope control device according to claim 12, wherein the endoscope information includes at least two of the insertion length, the insertion trajectory, the lesion position coordinates, the insertion portion distal end position coordinates, or the lesion observation direction.

14. The endoscope control device according to claim 1, wherein the first lesion identification determination based on the endoscope information is performed based on an insertion length of an insertion portion of the endoscope, an orientation of a distal end of the insertion portion of the endoscope, and lesion position coordinates.

15. The endoscope control device according to claim 1, wherein at least one of the one or more processors is configured to present positional relationship information between a currently observed site and an observation target region including a previously recognized lesion.

16. The endoscope control device according to claim 15, wherein at least one of the one or more processors is configured to generate operation information from information regarding the currently observed site and the observation target region including the previously recognized lesion.

17. An endoscope system comprising:
an endoscope; and
the endoscope control device according to claim 1.

18. An endoscope control method implementable on one or more processors of an endoscope control device, the endoscope control method comprising:
acquiring endoscope information and image information of an observation target region at a plurality of timings at which an endoscope is positioned in the observation target region; and
performing lesion identification determinations each determining whether lesions observed at the plurality of timings are identical to each other, thereby generating comparison information including one or more results derived from the lesion identification determinations, the lesion identification determinations comprising:
a first lesion identification determination based on the endoscope information;
a second lesion identification determination based on the image information; and
a third lesion identification determination based on results of the first lesion identification determination and the second lesion identification determination.

19. The endoscope control method according to claim 18, wherein the plurality of timings are timings at which the endoscope information is acquired in a same examination.

20. The endoscope control method according to claim 18, further comprising displaying the one or more results derived from the lesion identification determinations using a binary indication showing whether the lesions observed at the plurality of timings are identical to each other or different from each other or using a numerical value indicating reliability of the third lesion identification determination.
